(19) United States
(12) Patent Application Publication
(10) Pub. No.: US 2026/0301940 A1
(43) Pub. Date: Oct. 1, 2026

(54) AUTOMATED PERSONALIZED SELECTION OF INTRAOCULAR LENS

(71) Applicant: ALCON INC., Fribourg (CH)

(72) Inventors: Kevin BAKER, Duluth, GA (US); Lei SHI, Suwanee, GA (US); Somesh YADAV, Decatur, GA (US); Ramesh SARANGAPANI, Coppell, TX (US); George Hunter PETTIT, Fort Worth, TX (US); John Alfred CAMPIN, Southlake, TX (US); Sumit PALIWAL, Irvine, CA (US); Joseph WEATHERBEE, San Clemente, CA (US); Parvathy SARAT, Johns Creek, GA (US); Nicolette PARKINSON, Miami, FL (US); Elizabeth MIRANDA, Keller, TX (US)

(21) Appl. No.: 19/633,154

(22) Filed: Mar. 30, 2026

Related U.S. Application Data
(60) Provisional application No. 63/780,629, filed on Mar. 31, 2025.

(51) Int. Cl. G16H 40/67; G16H 10/60

(52) U.S. Cl. CPC G16H 40/67; G16H 10/60

(57) ABSTRACT

A system for selecting an intraocular lens includes a controller configured to selectively execute one or more machine learning models. The controller is configured to receive data from a smart device operable by the patient, the data being generated by an electronic virtual assistant configured to prompt the patient to answer one or more predefined questions at specific intervals and store respective answers entered by the patient. The controller is configured to present a plurality of trained models to a health care provider for selection. The trained models are adaptively trained with a respective training dataset drawn from at least one predefined category. The controller is configured to generate an automated recommendation for the intraocular lens from a group of intraocular lenses based in part on a plurality of input factors related to the patient, including self-reported data, and an output generated by the chosen machine learning model.

This application claims priority to U.S. Patent Application No. 63/780,629, filed Mar. 31, 2025, the entire contents of which are incorporated by reference in the present disclosure.

### INTRODUCTION

The disclosure relates generally to an automated system and method of selecting an intraocular lens for implantation in an eye. The human lens is generally transparent, such that light may travel through it with ease. However, many factors may cause areas in the lens to become cloudy and dense, and thus negatively impact vision quality. The situation may be remedied via a cataract procedure, whereby an artificial lens is selected for implantation into a patient's eye. Indeed, cataract surgery is a common surgery performed all around the world. An important clinical outcome driver for cataract surgery is the selection of an appropriate intraocular lens. Given the complexity and range of factors pertaining to the patient that must be taken into consideration, and the various types of intraocular lenses available in the market, it is challenging for providers to optimize selection of an appropriate intraocular lens.

### SUMMARY

Disclosed herein is a system and apparatus for selecting an intraocular lens for implantation into an eye of a patient. The selection may be based on candidate profile data corresponding to the patient. The candidate profile data may include one or more of lifestyle data, education data, clinical data, diagnostic data, demographic data, device use data, and genetic data, such that the selection may be informed by a comprehensive patient profile aggregating data from multiple data domains.

The above features and advantages and other features and advantages of the present disclosure are readily apparent from the following detailed description of the best modes for carrying out the disclosure when taken in connection with the accompanying drawings.

### BRIEF DESCRIPTION OF THE DRAWINGS

FIG. 1 is a schematic illustration of a system for selecting an intraocular lens for implantation into an eye using a model, the system having a controller;

FIG. 2 is a schematic flowchart for a selection method executable by the controller of FIG. 1;

FIG. 3 is a schematic diagram of an example group of intraocular lenses;

FIG. 4A shows schematic example illustrations of charts generated by the controller of FIG. 1, each chart having a comorbidity rule-out marker corresponding to a respective retinal surface;

FIG. 4B shows schematic example illustrations of charts generated by the controller of FIG. 1, each chart having a comorbidity rule-out marker corresponding to a respective corneal topography;

FIG. 5 is a schematic flowchart for a training method employable in the system of FIG. 1;

FIG. 6A is a schematic fragmentary cross-sectional view of an example pre-operative image of an eye;

FIG. 6B is a schematic fragmentary cross-sectional view of an example post-operative image of an eye;

FIG. 7 illustrates an example IOL selection system that may be used to select an IOL for a candidate patient that is a potential candidate for an IOL procedure;

FIG. 8 illustrates an example IOL outcome prediction process that may be used to generate an outcome prediction with respect to an IOL procedure for a candidate patient for the IOL procedure;

FIG. 9 illustrates a flowchart of an example method for determining an outcome prediction of an IOL procedure; and

FIG. 10 illustrates a flowchart of an example method for curating education materials for a candidate patient of an IOL procedure.

### DETAILED DESCRIPTION

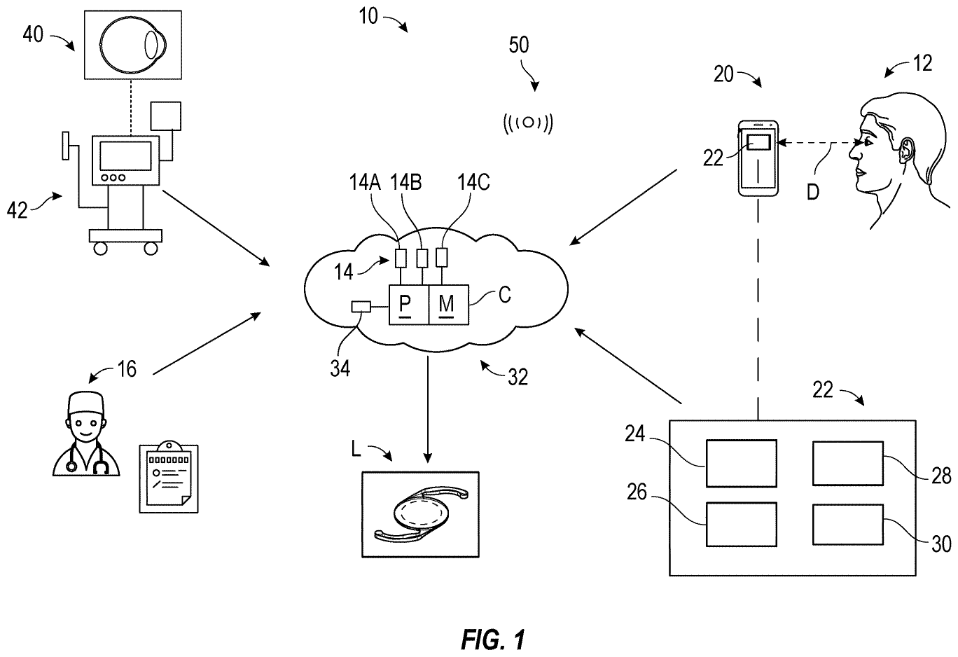

Referring to the drawings, wherein like reference numbers refer to like components, FIG. 1 schematically illustrates an automated selection apparatus or system 10 for selecting an intraocular lens L for implantation in a patient 12. Given the various types of intraocular lenses with specialized features available in the market (e.g., toric and presbyopia correcting), as well as the complexity and range of factors pertaining to the patient 12 that must be taken into consideration prior to a cataract procedure, it is challenging for health care providers to optimize selection of an appropriate intraocular lens.

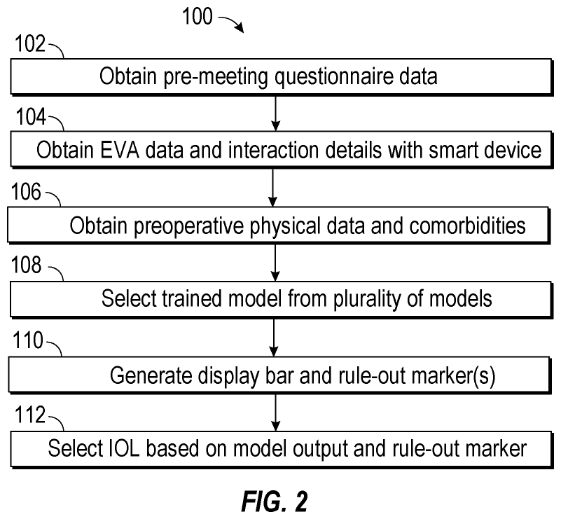

Referring to FIG. 1, the system 10 includes a controller C having at least one processor P and at least one memory M (or non-transitory, tangible computer readable storage medium) on which instructions are recorded for executing a method 100 for generating an automated recommendation for an intraocular lens L for the patient 12. Method 100 is shown in and described below with reference to FIG. 2.

The system 10 leverages various types of data for optimizing the selection process. The controller C is configured to present multiple trained models 14 (e.g., model 14A, model 14B, and model 14C) to a health care provider 16 (e.g., surgeon) for selection and selectively executes the chosen machine learning model. As described below, each of the model choices are adaptively trained with a respective training dataset drawn from at least one predefined category.

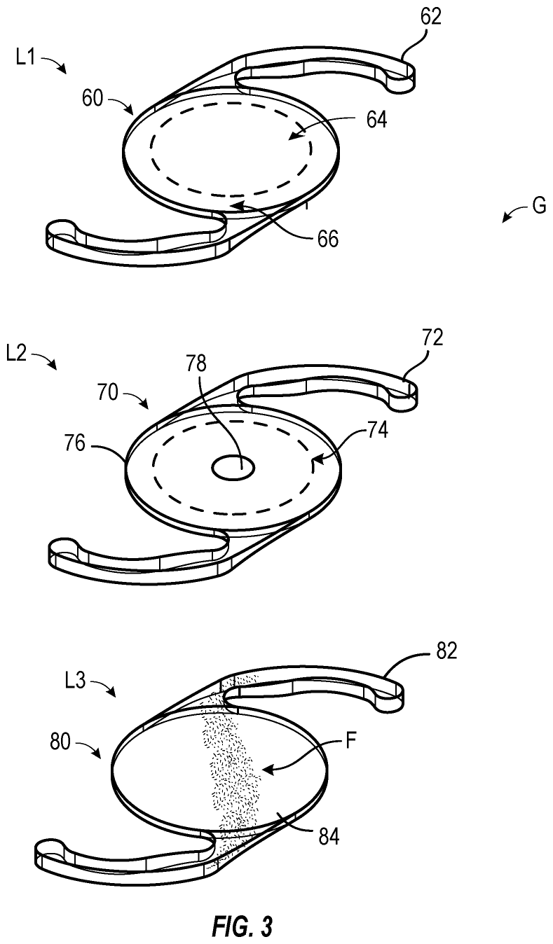

The system 10 generates an automated recommendation for the intraocular lens L from a group G of intraocular lenses (shown in FIG. 3) based in part on multiple input factors related to the patient 12, and an output generated by the chosen machine learning model. The automated recommendation is optimized for the type of intraocular lenses (e.g., monofocal, Vivity®, or PanOptix®), as well as other quantitative success criteria (e.g., likelihood of report by patient of post-op expectation of “does not meet,” “meets,” and “exceeds”, relative to expected improvement between pre-operative and post-operative results, e.g., for near and intermediate visual performance). The personalized suggestions for an intraocular lens L are based on the presence of ocular comorbidities (e.g., irregular corneal surface, retina diseases), lifestyle (e.g., time spent on digital devices, reading distance, time spent being active), expectations (e.g., spectacle independence motivation or patient motivation in general), and other factors.

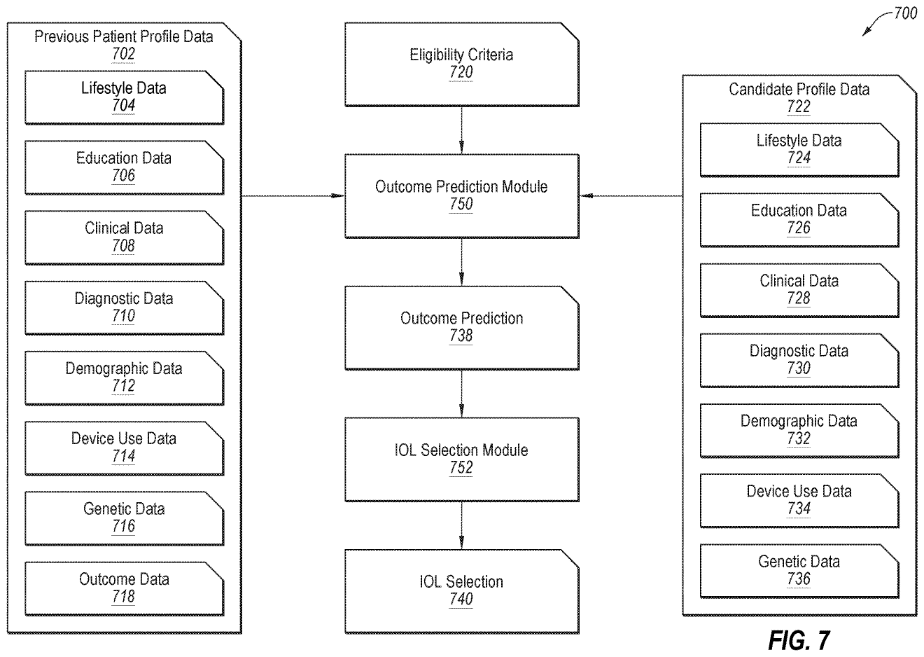

For example, in these and other embodiments, a comprehensive patient profile may be constructed for selection of the intraocular lens L. The patient profile may include information from different data domains corresponding to the patient. The data domains may include some of those already mentioned as well as others. For instance, in some embodiments, the data domains may include lifestyle information, patient education information, clinical information, diagnostic information, patient demographics, device use information, genetic information, and/or patient-reported outcomes. In these and other embodiments, the controller C may be configured to help select the intraocular lens L based on the patient profile. Additional information and details regarding the generation of the patient profile and selection of the intraocular lens L is given with respect to FIGS. 7-9.

Referring to FIG. 1, the controller C is configured to receive data from a smart device 20 operable by the patient 12. The smart device 20 is configured to communicate with, i.e. receive and transmit wireless communication, the controller C, via a network 50. The smart device 20 may be a smartphone, laptop, tablet, or other electronic device that the patient 12 may operate, for example with a touch screen interface or I/O device. The smart device 20 may include a respective processor and a respective memory. The smart device 20 may run an electronic virtual assistant 22, which may be included with or may be a mobile application or “app.” At least some data received from the smart device 20 may be obtained from user interactions with the app and/or may be generated by the electronic virtual assistant 22, which is configured to prompt the patient 12 to answer one or more predefined questions at specific intervals and store the respective answers entered by the patient 12 in response, as self-reported data. The electronic virtual assistant 22 may employ a pre-trained language model to generate conversational responses to questions posed by the patient 12 in real-time. Additionally or alternatively, the app may include one or more questionnaires for the patient to answer. The circuitry and components of a server, network and mobile application (“apps”) available to those skilled in the art may be employed. In these and other embodiments, the data received from the smart device 20 may include visual performance measurements of the patient 12 made with the smart device 20, becoming part of the multiple input factors. For example, the smart device 20 may be used to measure how much of a need a patient 12 has for presbyopia correcting lenses by measuring how close they hold the phone from their eye/face, or estimating their visual performance at near distances (e.g., through a mobile application asking them to read standard passages). Additional description about what may be included in the data obtained via the smart device 20 is described with respect to FIGS. 7-9. Further, although described as being obtained from the smart device, data used in the intraocular lens selection may be obtained via any other suitable device. Further, a website may be used to obtain the information rather than or in addition to a dedicated app. Additionally or alternatively, the electronic virtual assistant may be included with one or more of such websites.

Referring to FIG. 1, the controller C may be linked to a cloud database 32 with one or more servers hosted on the Internet to store, manage, and process data. The controller C may include a data management module 34 having a computerized data management system able to access information from the electronic medical records of various facilities. In some embodiments, the trained models 14 may be embedded in the controller C. Additionally or alternatively, the trained models 14 may be stored elsewhere and accessible to the controller C.

Referring now to FIG. 2, a flow chart of the method 100 is shown, which may be embodied as computer-readable code or instructions stored on and executable by the controller C of FIG. 1. Method 100 need not be applied in the specific order recited herein and some blocks may be omitted. The memory M can store controller-executable instruction sets, and the processor P can execute the controller-executable instruction sets stored in the memory M.

Per block 102 of FIG. 2, the method 100 includes obtaining pre-meeting questionnaire data. As shown in FIG. 1, the journey of a patient 12 may begin with filling out a questionnaire prior to meeting with the health care provider 16. The patient 12 is asked several lifestyle and expectation questions. These responses may be entered by the patient 12 into the smart device 20 (or any other suitable device) and may be transferred to the cloud database 32 that is linked or otherwise accessible to the controller C.

Per block 104 of FIG. 2, in some embodiments the controller C is configured to obtain data from the electronic virtual assistant 22 (EVA). The electronic virtual assistant 22 may be configured as a conversational artificial intelligence-based digital solution capable of answering a patient's questions on features of various intraocular lenses, cataract surgery info, etc. before consulting with their surgeon.

The electronic virtual assistant 22 is configured to prompt the patient to answer one or more predefined questions (e.g., displayed in box 24) at specific intervals, e.g. daily. The electronic virtual assistant is configured to store respective answers entered by the patient 12 (e.g., entered in box 26) in response to the questions, as self-reported data. The predefined questions may include an inquiry into a comfort level of the patient 12, such as a dryness factor and an irritation factor, including mined query data. Additionally or alternatively, the predefined questions may include questions regarding difficulty of a patient to see, haziness, cloudiness, or darkness of their vision, if certain times of the day or darkness levels aggravate the lack of visual acuity, and/or any other questions related to their vision.

The electronic virtual assistant 22 may answer questions posed by the patient 12 (e.g., entered in box 28). The patient 12 may enter follow up questions using the answers provided by the electronic virtual assistant 22. This ability of the electronic virtual assistant 22 to answer questions provides more data to support decision making. The questions to the electronic virtual assistant 22 posed by the patient 12 are entered as input factors to the model that will be chosen in block 108. In some embodiments, the electronic virtual assistant 22 may employ a natural language processing module to identify the most relevant sources of information based on the questions or queries of the patient 12 and provides these references to the patient 12 if they wish to read more on the topic. The contextual information may be obtained from trusted data sources (e.g., American Academy of Ophthalmology) that are relative authorities on cataract surgery, intraocular lens choices, etc. Relevant snippets from the content or files determined by the natural language processing module are used as context along with the specific questions asked by the patient 12. Finally, the context and patient question text may be sent to a predetermined language model to generate an automated response (e.g., displayed in box 30).

The intraocular lens L may be selected from a group G of intraocular lenses having particular features, such as a first intraocular lens L1, a second intraocular lens L2, and a third intraocular lens L3, shown in FIG. 3. It is to be understood that the intraocular lens L may take many different forms and include multiple and/or alternate components. Referring to FIG. 3, the first intraocular lens L1 includes an optic zone 60 contiguous with one or more supporting structures 62. The optic zone 60 may include an apodised diffractive multifocal zone 64 and an outer distance zone 66, with the first intraocular lens L1 being configured to provide an extended depth of focus, with a continuous range of vision from near to intermediate.

Referring to FIG. 3, the second intraocular lens L2 includes an optic zone 70 contiguous with one or more supporting structures 72. The optic zone 70 may include an apodised diffractive multifocal zone 74, an outer distance zone 76 and a center distance zone 78. The second intraocular lens L2 may be configured to provide crisper distance vision and improved intermediate vision, compared to the first intraocular lens L1. Referring to FIG. 3, the third intraocular lens L3 may be an accommodating lens with an optic zone 80 contiguous with one or more supporting structures 82. The intraocular lens L3 has an internal cavity 84 at least partially filled with fluid F. The fluid F is configured to be movable within the internal cavity 84 in order to vary the thickness (and power) of the third intraocular lens L3.

Although specific intraocular lenses of FIG. 3 are described, the present disclosure is not limited to selection from only these types of intraocular lenses. For example, the group G of intraocular lenses may include any other suitable types of intraocular lenses.

For instance, in some embodiments, the first lens L1 may be an extended depth of focus (EDOF) lens, such as the Alcon Vivity® intraocular lens. For example, the first lens L1 may include an optic zone having a non-diffractive, proprietary X-WAVE™ wavefront-shaping technology that stretches and shifts light rather than splitting it, thereby providing a continuous extended range of vision from intermediate to distance without the introduction of additional focal points. Additionally or alternatively, the first lens L1 may be configured to reduce the incidence of dysphotopsia, including halos and glare, relative to diffractive multifocal intraocular lenses, by virtue of its non-diffractive optical design. Further, the optic zone of the first lens L1 may include a central zone configured to apply the wavefront manipulation and a peripheral zone configured to contribute to distance vision correction. In addition, in some embodiments, the first lens L1 may be available in a toric configuration to address corneal astigmatism in addition to providing extended depth of focus.

Additionally or alternatively, the second lens L2 may be configured as a trifocal intraocular lens, such as the Alcon PanOptix® intraocular lens. For example, the second lens L2 may include an apodised diffractive optic zone configured to provide three distinct focal points corresponding to near, intermediate, and distance vision. Additionally or alternatively, the second lens L2 may be configured to utilize an ENLIGHTEN® optical technology that may direct a greater proportion of available light energy to the distance and intermediate focal points, thereby reducing the loss of light transmission that may otherwise be associated with trifocal diffractive designs. The second lens L2 may also be available in a toric configuration for patients presenting with corneal astigmatism.

Also, per block 104 of FIG. 2, the controller C obtains usage details of the patient 12 on the smart device 20, including a viewing distance D (see FIG. 1) between the patient 12 and the smart device 20. The viewing distance D may be measured along the line of sight of the patient 12 towards the screen of the smart device 20. The technical advantage provided by considering the viewing distance D is the optimization of the selected intraocular lens L for the viewing distance D that is most comfortable or most commonly used for the patient 12. In other words, the system 10 may give preference to selecting the intraocular lens L that provides crisper vision in the range of the viewing distance D.

Per block 106 of FIG. 2, the method 100 includes obtaining comorbidities data of the patient 12, which may be obtained during a consultation with the health care provider 16. The patient 12 would generally have answered the lifestyle and expectation questions before the cataract surgery planning meeting, and those responses may be automatically filled in during the consultation meeting. The patient 12 may change their responses during the consultation meeting if desired. In these and other embodiments, additional data may include an optical quality marker. In some embodiments, the optical quality marker may be derived from wavefront aberrometry data obtained from one or more of the imaging devices 42. Wavefront aberrometry may be configured to measure optical aberrations of the eye beyond simple sphere and cylinder corrections, including higher-order aberrations such as coma, spherical aberration, and trefoil. The optical quality marker may include a root mean square (RMS) wavefront error value, which may quantify the total wavefront error as a single numerical metric indicative of overall optical quality. Additionally or alternatively, the optical quality marker may include individual higher-order aberration coefficients corresponding to specific aberration modes, such as a spherical aberration coefficient or a coma coefficient. In these and other embodiments, the optical quality marker may be used as a gating criterion to determine whether the candidate patient may be suitable for premium IOL technologies that may require good baseline optical quality. For example, where the optical quality marker indicates a high level of higher-order aberrations or a high RMS wavefront error, the controller C may be configured to exclude certain presbyopia-correcting IOLs from consideration, as such IOLs may be more sensitive to pre-existing optical aberrations and may result in suboptimal visual outcomes in the presence of significant higher-order aberrations.

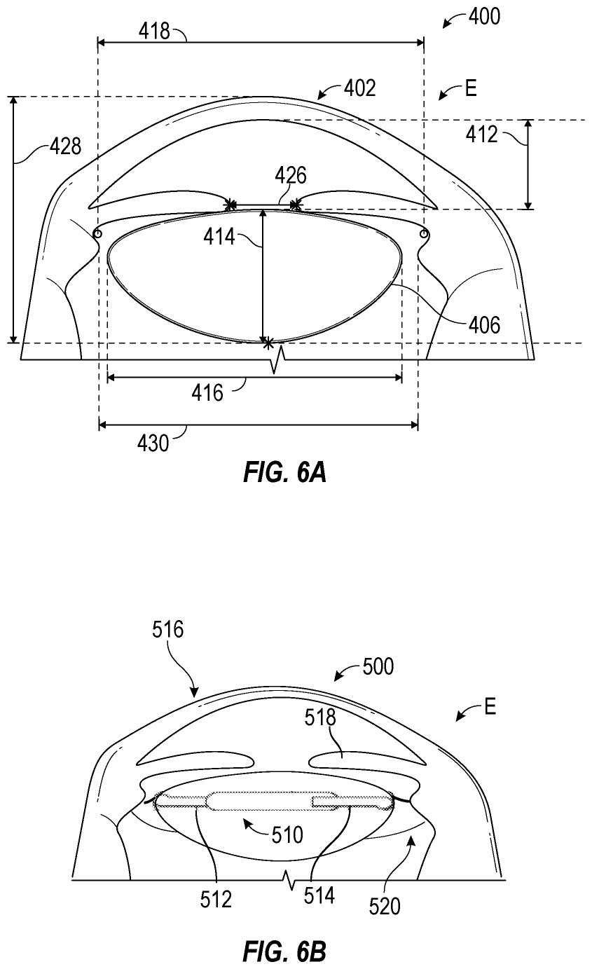

Also, per block 106 of FIG. 2, the controller C is adapted to obtain preoperative physical data 40, which may be from various imaging devices 42, such as corneal topography, tear film dynamics, wavefront, ultrasound, and/or optical coherence tomography (OCT) devices. The plurality of input factors may include the preoperative physical data 40 from at least one of the imaging devices 42, which includes at least one of a biometer, an OCT device, a camera system, or an aberrometry system. The preoperative physical data 40 may be in the form of a video, such as in the case of dynamic tear film surface measurements. It is to be understood that other imaging modalities may be employed. An example pre-operative image 400 of an eye E is shown in FIG. 6A. FIG. 6A is not drawn to scale. The pre-operative image 400 may be obtained via a biometer, an OCT device, a Scheimpflug camera, or an ultrasound bio-microscopy technique. The ultrasound bio-microscopy technique may employ a relatively high frequency transducer of between 35 MHz and 100 MHz, with a depth of tissue penetration between about 4 mm and 5 mm. Other imaging modalities may be employed, including but not limited to, OCT and magnetic resonance imaging.

Referring to FIG. 6A, the physical data may include pre-operative dimensions such as an anterior chamber depth 412, a lens thickness 414, a lens diameter 416 and/or a sulcus-to-sulcus diameter 418. Referring to FIG. 6A, the pre-operative dimensions may further include an iris diameter 426, an axial length 428 from the cornea 402 to a posterior surface of the pre-operative lens 406 and a ciliary process diameter 430. The preoperative physical data may include biometric parameters such as a K flat factor, a K steep factor, and an average K factor.

Per block 108 of FIG. 2, the method 100 includes presenting multiple trained models 14 (e.g., model 14A, model 14B, and/or model 14C shown in FIG. 1) to the health care provider 16 for selection. After the patient 12 and health care provider 16 have aligned on the lifestyle and expectation responses, the surgeon (or other health care provider) can decide which machine learning model they would like to use from a description of choices presented to them. The chosen machine learning model 14 is fed the self-reported data from the electronic virtual assistant 22 and other input factors from blocks 102, 104, 106.

In some embodiments, the machine learning model selection may be implied or derived based on other selections made by the health care provider. For example, in some embodiments, the health care provider may be given an option to select a particular IOL selection technique (e.g., algorithm) from a list of different IOL selection techniques or calculators. In some embodiments, the IOL selection calculators may include one or more of the following: a Barrett Universal II calculator, which uses theoretical optical formulas with personalized lens position prediction for IOL power calculation; a Hill-RBK calculator, which employs pattern recognition and artificial intelligence-based analysis using big data from previous cases; a Kane Formula, which combines theoretical optics with artificial intelligence and regression analysis for IOL power prediction; an EVO 2.0 calculator, which employs machine learning algorithms trained on large datasets to predict optimal IOL power; a Hoffer QST calculator, which is optimized for short eyes using adjusted anterior chamber depth predictions; a Holladay 2 calculator, which is a multi-variable formula incorporating multiple ocular parameters including white-to-white and lens thickness; a Haigis calculator, which uses three constants optimized for different axial lengths with anterior chamber depth as a key variable; a SRK/T calculator, which is a theoretical formula using axial length, keratometry, and A-constant for IOL power calculation; an Olsen calculator, which is a ray-tracing formula using thick lens theory and C-constant for precise optical modeling; and a Ladas Super Formula, which is an ensemble method combining multiple formulas with artificial intelligence weighting for improved accuracy.

At least some of the different selection techniques may use different machine learning models. As such, in some embodiments, selection of a particular selection technique may include the selection of a corresponding machine learning model.

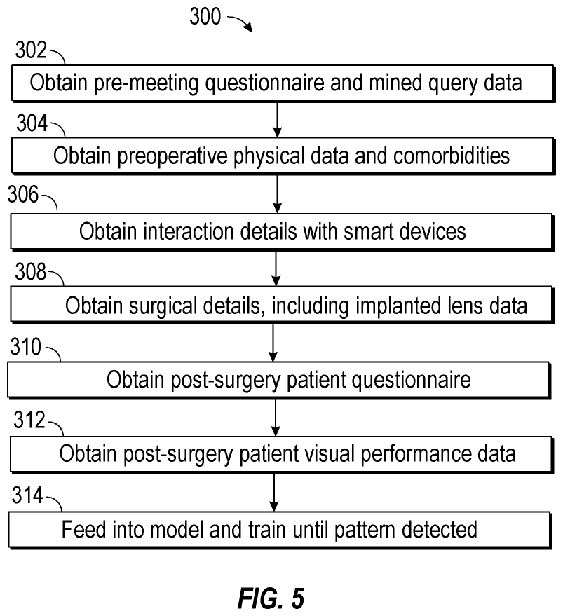

Each of the model choices may be adaptively trained with a respective training dataset drawn from at least one predefined category. An example training method 300 is described below with respect to FIG. 5. As described below with respect to FIG. 5, the training dataset includes respective historical sets for a large number of patients, including pre-operative objective data, pre-operative subjective data, intra-operative data, post-operative objective data, post-operative subjective data etc. for the large number of patients.

The predefined categories may include a specific health care provider such that the training dataset is drawn exclusively from respective patients of the specific health care provider (e.g., models 14A, 14B and 14C may be drawn from patients of Dr. John, Dr. Brown, and Dr. Smith, respectively). The predefined categories may include a specific facility such that the training dataset is drawn exclusively from respective patients associated with the specific facility. The predefined categories may include a specific region (e.g., a state or city) such that the training dataset is drawn exclusively from respective patients from the specific region. The predefined categories may include a specific demographic, such that the training dataset is drawn exclusively from respective patients of a certain age, gender, ethnicity, and/or socioeconomic status. The predefined categories may be mixed, e.g. models 14A, 14B and 14C may be drawn from a specific health care provider, facility, and region, respectively.

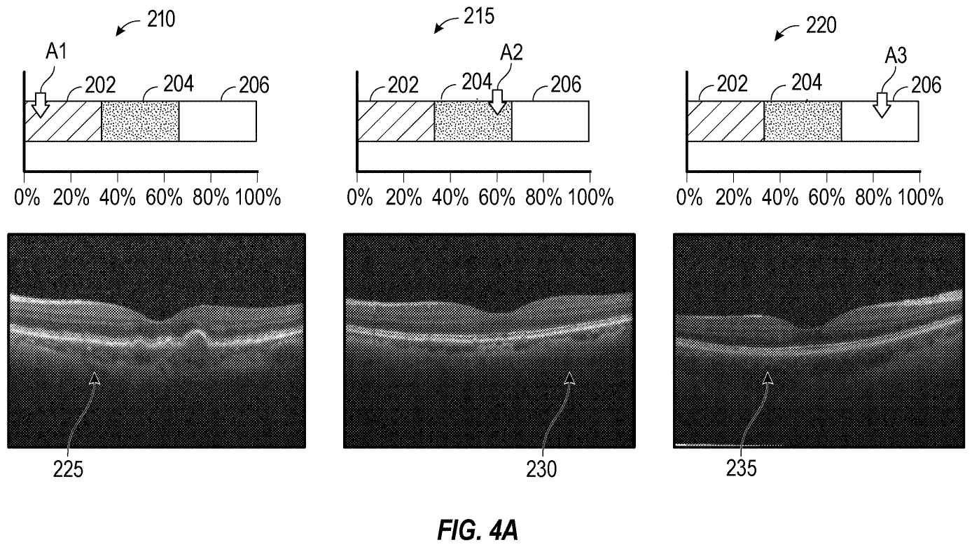

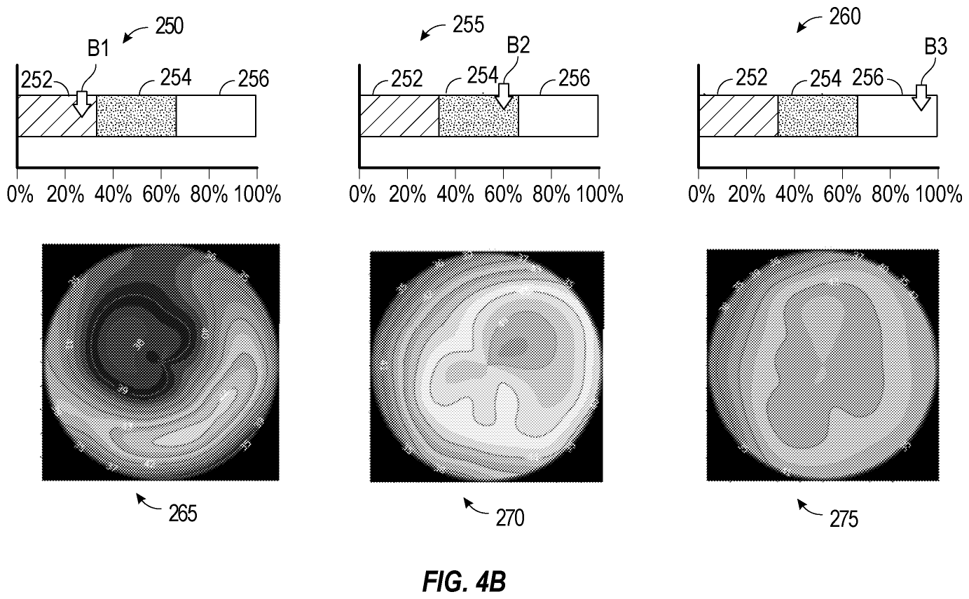

Per block 110 of FIG. 2, the controller C is configured to generate a display bar annotated with at least one comorbidity rule-out marker (e.g., comorbidity rule-out markers A1, A2, and A3 shown in FIG. 4A) for each intraocular lens in the group G based on the comorbidities data of the patient 12 obtained in block 106. It is understood that the display bar is presented as a non-limiting example and other forms of data display for the comorbidities data may be employed. FIG. 4A shows schematic examples of display bars 210, 215, 220 that are annotated with comorbidity rule-out markers A1, A2, and A3, respectively. Each display bar is respectively divided into a first portion 202, second portion 204, and third portion 206. The respective position of the comorbidity rule-out markers A1, A2, and A3 on the display bar 210, 215, 220 indicate the severity or magnitude of the comorbidity represented by the comorbidity rule-out marker (e.g., unacceptable, intermediate, acceptable). The comorbidity may be, for example, cornea curvature, eye dryness, retinal surface irregularity, and/or any other factors that may be related to ocular health.

In the example shown in FIG. 4A, the comorbidity under consideration is retinal surface irregularity, with the comorbidity rule-out markers A1, A2, and A3 corresponding to retinal surfaces 225, 230, 235, respectively. In some embodiments, the first portion 202, second portion 204, and third portion 206 are respectively displayed in red, yellow and green colors, similar to a traffic light. For example, comorbidity rule-out marker A1 falls in the first portion 202 (red) of the display bar 210, indicating that the intraocular lens at hand should be ruled out, or stated another way, because of the severity of the comorbidity (retinal surface irregularity), the associated intraocular lens is unsuitable for use. Comorbidity rule-out marker A3 is in the third portion 206 (green), indicating that the intraocular lens at hand is acceptable from a comorbidities point of view, or stated another way, because of the lightness of the comorbidity (retinal surface irregularity), the associated intraocular lens is well-suited for use. Comorbidity rule-out marker A2 is in the second portion 204 (yellow), indicating an intermediate position, or stated another way, because of the severity of the comorbidity (retinal surface irregularity), the associated intraocular lens is potentially suitable for use but should be selected with caution.

Similarly, FIG. 4B shows schematic examples of display bars 250, 255, 260 that are annotated with comorbidity rule-out markers B1, B2, and B3, respectively. Each display bar is respectively divided into a first portion 252, second portion 254, and third portion 256. In the example shown in FIG. 4B, the comorbidity under consideration is corneal surface irregularity, with the comorbidity rule-out markers B1, B2, and B3 corresponding to corneal topography surfaces 265, 270, 275, respectively. The respective position of the comorbidity rule-out markers B1, B2, and B3 on the display bar 250, 255, 260 indicates the severity or magnitude of the corneal surface irregularity (e.g., unacceptable, intermediate, acceptable). Additionally or alternatively, the comorbidity rule-out markers B1, B2, and B3 on the display bar 250, 255, 260 may indicate the acceptability level of a given intraocular lens based on the severity of the comorbidity (e.g., corneal surface irregularity).

In addition to the questionnaire responses, additional quantitative data may be used to help support the lens selection. For example, the smart device 20 may be employed to assess ocular comorbidities by capturing blink rates, eye redness, etc. associated with a patient's dry eye severity both before and/or after surgery. This may be employed in conjunction with the standard clinic measurements that measure various aspects of the cornea, retina, dry eye, etc. that are used to potentially rule out various intraocular lenses due to ocular comorbidities. Additionally or alternatively, such data may be useful in feeding back into the controller C to facilitate the intraocular lens L selection. For example, if a given patient had increased eye redness based on a selected intraocular lens and a future similar patient was concerned about or prone to eye redness, the controller C may be less likely to suggest the intraocular lens which was observed to induce eye redness. For example, the description related to FIGS. 7-9 illustrate additional manners in which the controller C may select a particular intraocular lens.

Per block 112 of FIG. 2, the controller C is configured to generate an automated recommendation for the intraocular lens L to be selected based on the output of the chosen model and the comorbidity rule-out marker(s) on the display bar. The recommendations may be weighted amongst the group G of intraocular lenses. In one example, the recommendation may be lens L1 with 50% strength, lens L2 and L3 with an equal suggestion weighting factor of 25%. In other words, after the health care provider 16 has decided which model to select (e.g., by selection of a particular IOL calculator), entered the input data into the model, and executed the program, the health care provider can review the weighted recommendations generated for each intraocular lens L in the group G. The intraocular lens having the highest weighting factor is selected by default for implantation. In some embodiments, such a selection may be overridden by the health care provider. In the case of multiple output factors, the controller C may be configured to use a weighted average of the multiple output factors or other statistical methods (e.g. a neural net).

Referring now to FIG. 5, a flow chart of a training method 300 employable by the system 10 is shown. Training method 300 need not be applied in the specific order recited herein and some blocks may be omitted. Per block 302 of FIG. 5, the training method 300 includes obtaining a pre-appointment lifestyle and expectation questionnaire, pre-meeting questionnaire, and/or mined query data from the electronic virtual assistant 22 for each patient in the training dataset.

Per block 304 of FIG. 5, the method 300 includes obtaining preoperative physical data and/or comorbidities data of each patient in the training dataset. An example pre-operative image 400 of an eye E is shown in FIG. 6A. The comorbidity data may be obtained during the planning meeting between the respective patient and the health care provider 16. The prior responses from block 302 can be combined with ocular comorbidity data/diagnoses (e.g., dry eye, retinal diseases) and saved as input data.

Per block 306 of FIG. 5, the controller C is configured to obtain interaction details with a smart device of the patient 12, including a respective viewing distance D of the respective patient in the training dataset from the smart device 20.

Per block 308 of FIG. 5, the method 300 includes obtaining surgical details or intra-operative data with information related to the actual treatment performed. This information may be captured electronically and fed into the data management module 34. Examples of intra-operative data include, but are not limited to, the type of refractive surgery procedure performed, the model of the implanted intraocular lens and its prescription. The intra-operative data may include intra-operative aberrometry measurements. The intra-operative data may further include the surgical machine settings and parameters of the procedure, such as procedure time, the temperature of the operating room, the total phaco power consumed to emulsify the original lens, the time duration that the phaco energy was applied, and the effective phaco time (as a product of phaco time multiplied by an average phaco power). The intra-operative data may further include: the type of delivery device used to implant the intraocular lens, the presence or absence of any occlusion breaks, the quantity and degree of the occlusion breaks, and whether or not assistive devices (such as capsular hooks) were employed. The intra-operative data may further include an intra-operative grade of nuclear hardness of the original lens, which may be graded according to a lens opacity classification.

Per block 310 of FIG. 5, the controller C is configured to obtain post-surgery patient questionnaire data. For example, after surgery, each patient in the training dataset may be requested to provide information on their satisfaction (e.g., does not meet expectations, meets expectations, exceeds expectations, less time spent wearing glasses, etc.). Additional quantitative criteria may be used to assess success (e.g., vision performance at near, intermediate, and distance; time spent not wearing reading glasses; time spent doing physical activities, hobbies, etc.).

Per block 312 of FIG. 5, the method 300 includes obtaining quantitative patient visual performance measurements. The quantitative patient visual performance measures may be measured by the health care provider 16 post-operatively. The visual performance measurements may be measured with a mobile device (e.g., answers to subjective questions, images captured or measured with the mobile device), and/or clinic instruments. The visual performance measurements may include, among other things, visual acuity (e.g., the ability to see fine details at a specific distance), contrast sensitivity (e.g., how well someone can distinguish between objects with different levels of contrast against their background), and the presence of visual disturbances. Block 312 includes obtaining post-operative imaging data. An example post-operative image 500 is shown in FIG. 6B and may be obtained via a biometer, an OCT, or other imaging modality available to those skilled in the art. FIG. 6B illustrates the implanted intraocular lens 510, the first supporting structure 512, the second supporting structure 514, the cornea 516, the iris 518 and the ciliary muscle 520. FIG. 6B is not drawn to scale.

Per block 314 of FIG. 5, the data obtained in blocks 302, 304, 306, 308, 310, and/or 312 are fed into the program until a pattern is detected. In some embodiments, the predefined model category is a specific heath care provider. Here, once the machine learning model has learned the pattern on how the health care provider 16 (e.g. a specific surgeon) is making their decisions, the automated solution will inform the health care provider 16 and allow them to save this model so that it can either be used by their colleagues or to provide subsequent intraocular lens recommendations.

The machine learning models (e.g., decision trees) may be configured to learn from each provider's specific selection criteria and seek desirable post-operative outcomes (e.g., high patient satisfaction). Additionally, these models help to enable a health care provider 16 with less experience to gain more confidence suggesting specialized intraocular lenses (e.g., monofocal, Vivity®, or PanOptix®) by identifying which patients are more likely to be highly satisfied with them. The machine learning models may be configured to find parameters, weights, or a structure that minimizes a respective cost function. Each of the machine learning models may be a respective regression model. It is understood that other types of machine learning models available to those skilled in the art may be employed.

In some embodiments, the machine learning models are or include decision trees. As understood by those skilled in the art, a decision tree is a supervised learning algorithm that is nonparametric. It has a hierarchical tree structure, which includes a root node, branches, internal nodes, and leaf nodes. The machine language model decision tree identifies a series of variables that affect the outcome of a situation. Each variable is assigned a relative weight, with the higher weights having a greater impact on the outcome. Once the variables are set, the process is carried forward in each branch until a final conclusion is reached. After many iterations, a decision tree will be trained on the actual intraocular lenses that were implanted, the factors associated with those cases, and/or the observed outcomes. In some embodiments, the controller C may be configured to minimize a cost function defined as the mean squared error between a predicted manifest refraction spherical equivalent (based on the pre-operative image 400) and a post-operative manifest refraction spherical equivalent (based on the post-operative image 500).

In some embodiments, the patient selection algorithms may be based on generative artificial intelligence (AI) models. Generative AI refers to artificial intelligence systems capable of creating new content, such as text, images, audio, or code, by learning patterns from vast datasets. Generative AI works by training advanced machine learning models, such as neural networks, on large datasets to learn patterns, structures, and relationships in the data. In one example, multi-model data inputs (e.g., patient survey questionnaires, corneal topographies, retina surface scans, etc.) are fed into a generative AI-based patient selection algorithm that provides a comprehensive response such as: “Based on the patient's corneal topography there is an 80% likelihood that they will have an acceptable post-operative outcome with a PanOptix® IOL. Furthermore, the patient's unique lifestyle that includes limited driving at night and hobbies requiring near work indicate that a PanOptix® IOL would best suit their unique lifestyle”.

In summary, the selection system 10 optimizes, enhances, or improves the selection process for an intraocular lens L. The controller C is configured to selectively execute a machine learning model. The machine learning model is adaptively trained with a training dataset drawn from at least one predefined category. The machine learning models may learn from the choices made by specific surgeons or providers with patient data (e.g., comorbidities, lifestyle, expectations, post-operative satisfaction, etc.) and are used to minimize or reduce time and resources spent making future recommendations.

The system 10 may provide the following technical advantages: (i) provide intraocular lens recommendations (e.g., toric, presbyopia correcting, monofocal) based on patient-centric data and learning from specific training datasets; (ii) leverage expertise of numerous expert surgeons when making decisions; (iii) help health care providers decide what intraocular lens to implant for patients that are more difficult to evaluate; (iv) help experienced health care providers understand patient types that may benefit from recently available intraocular lenses; (v) prior to surgery, predict patient post-operative expectations (e.g., satisfaction) with various intraocular lenses; and (vi) obtain quantitative assessment of ocular comorbidities likely to rule-out premium intraocular lenses regardless of other factors.

The machine learning models of FIG. 1 may include a neural network algorithm. As understood by those skilled in the art, neural networks are designed to recognize patterns and may be modeled loosely after the human brain. The patterns are recognized by the neural networks from real-world data (e.g. images, sound, text, time series, and others) that is translated or converted into numerical form and embedded in vectors or matrices. The neural network may employ deep learning maps to match an input vector x to an output vector y. Stated differently, each of the machine learning models learns an activation function f such that f(x) maps to y. The training process enables the neural network to correlate the appropriate activation function f(x) for transforming the input vector x to the output vector y. In the case of a simple linear regression model, two parameters are learned: a bias and a slope. The bias is the level of the output vector y when the input vector x is 0 and the slope is the rate of predicted increase or decrease in the output vector y for each unit increase in the input vector x. Once the machine learning models are respectively trained, estimated values of the output vector y may be computed with given new values of the input vector x.

Referring to FIG. 1, the network 50 may be a Wireless Local Area Network (LAN) which links multiple devices using a wireless distribution method, a Wireless Metropolitan Area Networks (MAN) which connects several wireless LANs or a Wireless Wide Area Network (WAN) which covers large areas such as neighboring towns and cities. Other types of connections may be employed. The network 50 may be a bus implemented in various ways, such as for example, a serial communication bus in the form of a local area network. The local area network may include, but is not limited to, a Controller Area Network (CAN), a Controller Area Network with Flexible Data Rate (CAN-FD), Ethernet, blue tooth, WIFI and other forms of data connection.

The controller C of FIG. 1 includes a computer-readable medium (also referred to as a processor-readable medium), including a non-transitory (e.g., tangible) medium that participates in providing data (e.g., instructions) that may be read by a computer (e.g., by a computer processor). Such a medium may take many forms, including, but not limited to, non-volatile media and volatile media. Non-volatile media may include, for example, optical or magnetic disks and other persistent memory. Volatile media may include, for example, dynamic random-access memory (DRAM), which may constitute a main memory. Such instructions may be transmitted by one or more transmission media, including coaxial cables, copper wire and fiber optics, including the wires that comprise a system bus coupled to a processor of a computer. Some forms of computer-readable media include, for example, a floppy disk, a flexible disk, hard disk, magnetic tape, other magnetic medium, a CD-ROM, DVD, other optical medium, other physical medium with patterns of holes, a RAM, a PROM, an EPROM, a FLASH-EEPROM, other memory chip or cartridge, or other medium from which a computer can read.

Look-up tables, databases, data repositories or other data stores described herein may include various kinds of mechanisms for storing, accessing, and retrieving various kinds of data, including a hierarchical database, a set of files in a file system, an application database in a proprietary format, a relational database management system (RDBMS), etc. Each such data store may be included within a computing device employing a computer operating system such as one of those mentioned above, and may be accessed via a network in one or more of a variety of manners. A file system may be accessible from a computer operating system and may include files stored in various formats. An RDBMS may employ the Structured Query Language (SQL) in addition to a language for creating, storing, editing, and executing stored procedures, such as the PL/SQL language mentioned above.

Referring now to FIG. 7, FIG. 7 illustrates an example IOL selection system 700 (“system 700”) that may be used to select an IOL for a candidate patient that is a potential candidate for an IOL procedure. The system 700 may be arranged in accordance with at least one embodiment described in the present disclosure. In some embodiments, the system 700 may be part of and/or implemented by any of the systems or devices described in this disclosure. For example, the system 700 may be part of and/or implemented by the controller C of FIG. 1.

In general, the system 700 may be configured to apply a holistic analysis of a candidate patient and previous patients to determine an outcome prediction 738. In these and other embodiments, the outcome prediction may be used to inform an IOL selection 740. For example, the system 700 may be configured to construct comprehensive patient profiles by aggregating data from multiple data domains and to determine the outcome prediction 738 based on a holistic analysis of the candidate patient compared against previous patients. This holistic approach may improve prediction accuracy by accounting for factors beyond anatomical measurements that may influence patient satisfaction and clinical outcomes. By comparison, conventional IOL selection approaches may rely on a more limited data domain that primarily includes clinical measurements and diagnostic imaging obtained during pre-operative examinations, which may provide an incomplete picture of patient suitability and expected outcomes.

For ease of explanation, the system 700 is described in relation to “modules” that perform certain operations or functions. Various functions described herein as being performed by “modules” may be carried out by hardware, firmware, and/or software. For instance, various functions may be carried out using one or more processors (e.g., central processing units (CPU(s)), graphics processing units (GPU(s)), microprocessors, microcontrollers, embedded processors, digital signal processors (DSPs), image signal processors (ISPs), physics processing units (PPUs), field-programmable gate arrays (FPGAs), accelerator(s) (e.g., deep learning accelerators (DLAs), deep learning accelerator cluster (XNNs), neural network accelerators (NNAs), and/or neural processing units (NPUs), programmable vision accelerators (PVAs), optical flow accelerators (OFAs), etc.), application specific integrated circuits (ASICs), data processing units (DPUs), quantum processors, etc.) executing instructions stored in memory. In these and other embodiments, one or more of the modules may employ or include one or more artificial intelligence (AI) models to process input information and generate one or more inference outputs accordingly. The AI models may include machine learning models, deep learning models, neural networks, or any other suitable models capable of learning patterns from data and making predictions or classifications based on learned patterns. In these and other embodiments, the AI models may be trained using historical data corresponding to prior IOL patients who have undergone an IOL procedure.

The system 700 may include an outcome prediction module 750 (“prediction module 750”) in some embodiments. The prediction module 750 may be configured to generate an outcome prediction 738 for the candidate patient based on previous patient profile data 702, one or more eligibility criteria 720, and/or candidate profile data 722.

The previous patient profile data 702 may include data corresponding to the prior IOL patients who have previously undergone an IOL related procedure. The previous patient profile data 702 may indicate individual profiles of the previous IOL patients in which the profiles include data corresponding to different domains that may be useful in determining the outcome prediction 738. Although not explicitly illustrated, the previous patient profile data 702 may be organized according to the patient profiles such that each previous IOL patient has a profile corresponding thereto and such that each patient profile includes data from the different data domains that corresponds to the respective previous IOL patient.

In some embodiments, a data domain of the previous patient profile data 702 may include lifestyle data 704. The lifestyle data 704 may represent a data domain within the previous patient profile data 702 that captures behavioral and environmental factors of the previous IOL patients. For example, the lifestyle data 704 may include information pertaining to the daily habits, activities, and behavioral patterns of the previous IOL patients that may have influenced their respective IOL outcomes and satisfaction levels.

In these and other embodiments, the lifestyle data 704 may include digital device usage information corresponding to previous IOL patients. For example, the lifestyle data 704 may include data indicating the number of hours per day a previous IOL patient spent using a computer, smartphone, or tablet. Additionally or alternatively, the lifestyle data 704 may further include data indicating the viewing distance D at which a previous IOL patient held a smart device 20, such as a smartphone, relative to their eye. This viewing distance information may be indicative of the near vision demands of the previous IOL patient. In these and other embodiments, the near vision demands may be used to identify cohorts of previous IOL patients with similar near vision usage patterns to the candidate patient.

Additionally or alternatively, the lifestyle data 704 may include occupational demand information corresponding to previous IOL patients. For example, the lifestyle data 704 may include data indicating whether a previous IOL patient performed sustained near work tasks, such as reading or detailed close-range work, as part of their occupation. The lifestyle data 704 may further include data indicating whether a previous IOL patient performed night work or work under low-light conditions.

In these and other embodiments, the lifestyle data 704 may include recreational activity information corresponding to previous IOL patients. For example, the lifestyle data 704 may include data indicating the types of hobbies and physical activities engaged in by previous IOL patients, such as outdoor sports, driving, reading, or other activities that place specific demands on near, intermediate, or distance vision. The lifestyle data 704 may further include data indicating the frequency and duration of such recreational activities.

Additionally or alternatively, the lifestyle data 704 may include personality and behavioral characteristic information corresponding to previous IOL patients. For example, the lifestyle data 704 may include data indicating whether a previous IOL patient exhibited detail-oriented or perfectionistic behavioral tendencies, which may be relevant to predicting tolerance for optical compromises associated with certain IOL types. The lifestyle data 704 may further include data indicating the degree to which a previous IOL patient expressed motivation for spectacle independence, which may be relevant to predicting post-operative satisfaction.

In some embodiments, the lifestyle data 704 may include a computed lifestyle risk score corresponding to each previous IOL patient. The lifestyle risk score may be computed as a weighted sum of lifestyle sub-factors, including digital device hours normalized to a waking day, occupational demand metrics, recreational activity indicators, personality, behavioral indicators, and/or an inverse wellness score.

In these and other embodiments, the different factors may be weighted differently in the generation of the lifestyle risk score. In some embodiments, the weighting comes as a result of AI algorithm development. Through the different historical data, the system may learn which factors have a stronger weighting. For example, the amount of time spent on near tasks or the computer might have a higher weighting to Multifocal suitability compared to time spent watching TV which might have a lower weighting.

In some embodiments, the lifestyle risk score may be expressible as:

$${L}_{r i s k} = {w}_{1} ( digital\_hours / 16 ) + … + {w}_{5} ( welllness\_score \_inverse ) .$$

The lifestyle risk score may accordingly serve as a composite numerical representation of the lifestyle domain for a given previous IOL patient, enabling quantitative comparison of lifestyle profiles between previous IOL patients and the candidate patient in some embodiments.

In some embodiments, the lifestyle data 704 may include self-reported data generated by the electronic virtual assistant 22 of the smart device 20 operated by previous IOL patients. For example, the lifestyle data 704 may include responses provided by previous IOL patients to predefined questions posed by the electronic virtual assistant 22, including questions regarding daily visual activities, comfort levels, and behavioral habits. Additionally or alternatively, the lifestyle data 704 may also include objective behavioral data retrieved from mobile health applications associated with the smart devices 20 of previous IOL patients, such as screen time logs, activity logs, display brightness adjustment histories, and font size adjustment histories, each of which may serve as an objective indicator of the visual demands and behavioral patterns of the previous IOL patient.

In some embodiments, another data domain of the previous patient profile data 702 may include education data 706. The education data 706 may represent a data domain within the previous patient profile data 702 that captures the level of comprehension and health literacy of the previous IOL patients. For example, the education data 706 may include information pertaining to the degree to which previous IOL patients understood the nature of their ocular condition, the available IOL options, and the expected outcomes associated with each IOL type prior to and following their respective IOL procedures. In these and other embodiments, the education data 706 may indicate the level of patient education of the previous IOL patients, including the extent to which a given previous IOL patient demonstrated comprehension of the technology associated with their selected IOL, the extent to which a given previous IOL patient demonstrated general health literacy, and the effectiveness with which a given previous IOL patient communicated their visual needs and expectations to their health care provider.

In some embodiments, the education data 706 may be derived from responses provided by previous IOL patients to structured questionnaires administered at one or more points along the pre-operative patient journey. For example, the education data 706 may be sourced from responses entered by previous IOL patients into the smart device 20 via the electronic virtual assistant 22. The electronic virtual assistant 22 may be configured to pose predefined questions to previous IOL patients at specific intervals, and the responses entered by those patients may be stored as self-reported data that reflects the patient's level of understanding of their condition and treatment options. In these and other embodiments, the education data 706 may further be sourced from queries posed by previous IOL patients to the electronic virtual assistant 22, wherein the nature and complexity of those queries may serve as an indicator of the patient's baseline level of education with respect to IOL selection and cataract surgery.

In some embodiments, the education data 706 may include an Education Understanding Score computed for individual previous IOL patients. The Education Understanding Score may be computed as a weighted composite of sub-scores corresponding to technology comprehension, health literacy, and communication effectiveness in some embodiments. For example, the Education Understanding Score may be expressible as:

$$E = {w}_{1} \cdot Etect + {w}_{2} \cdot Ehealth + {w}_{3} \cdot Ecomm ,$$

In the above expression, Etech represents the degree to which the previous IOL patient demonstrated comprehension of the specific IOL technology relevant to their procedure, Ehealth represents the general health literacy level of the previous IOL patient, and Ecomm represents the effectiveness with which the previous IOL patient communicated their visual needs and expectations. The weights “w” may indicate different weights that may be applied to the different education score factors. For example, watching educational content completely versus only watching 50% of it may give the literacy a higher weighting in the overall multifocal suitability score. Additionally or alternatively, the education presentation may include a “check for understanding” step at the end. A patient who scored 100% might have their health literacy provide a higher weighting in the overall suitability score than someone who only scored 50% correct or did not complete it at all. In these and other embodiments, the Education Understanding Score may serve as a composite numerical representation of the education domain for a given previous IOL patient, enabling quantitative comparison of education profiles between previous IOL patients and the candidate patient.

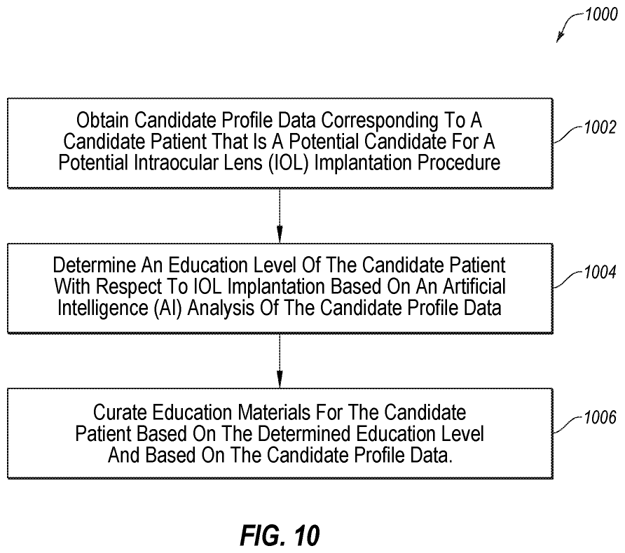

In some embodiments, the education data 706 may further include data indicating the modality through which educational content was delivered to individual previous IOL patients. For example, the education data 706 may indicate whether a previous IOL patient received educational content via the mobile application on the smart device 20, via a web-based portal, or via a link-based access mechanism delivered through an SMS text message or email. The education data 706 may further indicate the degree to which a previous IOL patient engaged with the educational content delivered to them, such as the duration of engagement, the number of content items reviewed, and whether the patient sought additional information by posing follow-up questions to the electronic virtual assistant 22 or otherwise seeking additional information (e.g., entering a search query into a Frequently Asked Questions database). In these and other embodiments, the education data 706 may indicate whether the educational materials were curated for the particular previous IOL patient, such as described with respect to FIG. 10 in the present disclosure.

In these and other embodiments, the education data 706 may include data indicating the level of patient education as it relates to post-operative outcomes. For example, the education data 706 may include data indicating whether a previous IOL patient with a higher Education Understanding Score demonstrated greater compliance with post-operative care instructions, greater tolerance for optical compromises associated with certain IOL types, or higher levels of post-operative satisfaction relative to previous IOL patients with lower Education Understanding Scores.

In some embodiments, another data domain of the previous patient profile data 702 may include clinical data 708. The clinical data 708 may represent a data domain that captures objective clinical measurements and assessments obtained from previous IOL patients during their respective pre-operative evaluations. For example, the clinical data 708 may include information pertaining to the ocular health status, medical history, and clinical findings of the previous IOL patients that may have influenced their respective IOL outcomes and satisfaction levels.

In these and other embodiments, the clinical data 708 may be sourced from electronic health records of previous IOL patients. For example, the clinical data 708 may include data retrieved from EHR systems associated with the health care provider 16 or with one or more clinical facilities at which previous IOL patients received care. The data management module 34 of the controller C may be configured to access and retrieve the clinical data 708 from the electronic medical records of various facilities.

In these and other embodiments, the clinical data 708 may include data pertaining to the prior surgical history of previous IOL patients. For example, the clinical data 708 may include data indicating whether a previous IOL patient had undergone prior laser vision correction procedures, such as LASIK or PRK, prior to their IOL implantation procedure. Such prior refractive surgical history may be relevant to IOL power calculation and lens selection, and may accordingly influence the outcome prediction 738 generated for the candidate patient. In some embodiments, the clinical data 708 may further include data indicating the presence or absence of ocular comorbidities in previous IOL patients, such as glaucoma, retinal pathology, or corneal irregularities, each of which may have influenced the IOL selection and post-operative outcomes of the respective previous IOL patient.

In these and other embodiments, the clinical data 708 may include data pertaining to the in-clinic medical and ocular history collected during direct patient interviews conducted by the health care provider 16 or clinic staff. In these and other embodiments, the clinical data 708 may be obtained in response to predefined questions posed by the electronic virtual assistant 22 regarding ocular health status and medical history.

In some embodiments, another data domain of the previous patient profile data 702 may include diagnostic data 710. The diagnostic data 710 may represent objective measurements and assessments of the ocular anatomy and physiology of the previous IOL patients. In some embodiments, the diagnostic data 710 may include imaging data obtained from one or more of the imaging devices 42 described above with respect to FIG. 1. For example, the diagnostic data 710 may include corneal topography data, wavefront aberrometry data, optical coherence tomography (OCT) data, ultrasound biomicroscopy data, optical biometry data, and/or tear film dynamics data corresponding to previous IOL patients. The diagnostic data 710 may further include biometric parameters such as axial length measurements, anterior chamber depth measurements, lens thickness measurements, keratometry values, and other anatomical parameters of the eye that may have influenced the IOL selection and post-operative outcomes of the respective previous IOL patient. In some embodiments, the diagnostic data 710 may include pre-operative image data, such as the pre-operative image 400 of the eye E shown in FIG. 6A. The diagnostic data 710 may further include post-operative imaging data, such as the post-operative image 500 shown in FIG. 6B. In some embodiments, the diagnostic data 710 may include video data, such as dynamic tear film surface measurement data captured in video form.

In some embodiments, the diagnostic data 710 may be sourced from the imaging devices 42 of FIG. 1. Additionally or alternatively, the diagnostic data 710 may be retrieved by the data management module 34 of the controller C from electronic medical records of various clinical facilities. In these and other embodiments, the diagnostic data 710 may be obtained during the pre-operative evaluation of the respective previous IOL patient, at which time the imaging devices 42 may be employed to capture the relevant ocular measurements and imaging data. The diagnostic data 710 may further be obtained from intra-operative measurements, such as intra-operative aberrometry measurements of the type described above with respect to block 308 of FIG. 5. In some embodiments, the diagnostic data 710 may also include post-operative imaging data obtained following the IOL implantation procedure of the respective previous IOL patient, such as the post-operative image 500 of FIG. 6B.

In some embodiments, another data domain of the previous patient profile data 702 may include demographic data 712. The demographic data 712 may represent population-level characteristics of the previous IOL patients. For example, the demographic data 712 may include information pertaining to the age, gender, ethnicity, and socioeconomic status of the previous IOL patients. In these and other embodiments, the demographic data 712 may further include geographic information corresponding to previous IOL patients, such as the region, state, or city in which a previous IOL patient resided at the time of their IOL procedure. The demographic data 712 may further include occupational classification data corresponding to previous IOL patients. In some embodiments, the occupational classification data may serve as a proxy indicator of socioeconomic status and near vision occupational demands.

In these and other embodiments, the demographic data 712 may include patient motivation data corresponding to previous IOL patients. For example, the demographic data 712 may include data indicating the degree to which a previous IOL patient expressed motivation for spectacle independence, which may be relevant to predicting post-operative satisfaction with a given IOL type. The demographic data 712 may further include data indicating the general health and wellness status of previous IOL patients, including body mass index, systemic health conditions, and other population-level health indicators that may have influenced IOL outcomes.

The demographic data 712 may be sourced from multiple origins. For example, the demographic data 712 may be retrieved by the data management module 34 of the controller C from electronic health records of various clinical facilities. Additionally or alternatively, the demographic data 712 may be sourced from responses provided by previous IOL patients to structured questionnaires administered through the patient-facing interface, such as the smart device 20. In these and other embodiments, the demographic data 712 may be sourced from responses entered by previous IOL patients into the smart device 20 via the electronic virtual assistant 22. The demographic data 712 may further be sourced from intake forms completed by previous IOL patients at the clinical facility at which their IOL procedure was performed.

In some embodiments, another data domain of the previous patient profile data 702 may include device use data 714. The device use data 714 may represent behavioral and interaction patterns of previous IOL patients with their personal electronic devices. For example, the device use data 714 may include data pertaining to the manner in which previous IOL patients interacted with the smart device 20 during the pre-operative period. In these and other embodiments, the device use data 714 may include data indicating the viewing distance D at which a previous IOL patient held the smart device 20 relative to their eye. The device use data 714 may further include data indicating the font size settings employed by a previous IOL patient on the smart device 20, which may serve as an objective indicator of the near vision demands of that patient. The device use data 714 may further include data indicating the display brightness level adjustments made by a previous IOL patient on the smart device 20 over time, which may be indicative of progressive changes in visual function associated with cataract development. The device use data 714 may further include screen time duration data corresponding to previous IOL patients, indicating the total amount of time spent interacting with the smart device 20 on a daily basis.

In these and other embodiments, the device use data 714 may be sourced from the smart device 20 operated by previous IOL patients. For example, the device use data 714 may be retrieved from mobile health applications associated with the smart devices 20 of previous IOL patients, such as screen time logs, display brightness adjustment histories, and font size adjustment histories. In some embodiments, the device use data 714 may be sourced from objective behavioral data retrieved via mobile health data platform integrations, such as application programming interface connections to health data aggregation platforms accessible through the smart device 20. Additionally or alternatively, the device use data 714 may be sourced from data generated by the electronic virtual assistant 22 operating on the smart device 20 of previous IOL patients. For example, the electronic virtual assistant 22 may be configured to record and transmit interaction data corresponding to the manner in which previous IOL patients engaged with the smart device 20, including the viewing distance D measured during sessions with the electronic virtual assistant 22.

In some embodiments, the device use data 714 may be included in the lifestyle data 704. For example, the digital device usage information captured within the device use data 714, such as the number of hours per day a previous IOL patient spent using the smart device 20 and the viewing distance D at which the smart device 20 was held, may be incorporated into the lifestyle data 704 as a component of the digital device usage information described above. Additionally or alternatively, the device use data 714 may be used to determine the lifestyle data 704. For example, the viewing distance D data and screen time duration data contained within the device use data 714 may be processed to derive lifestyle sub-factors that contribute to the computation of the lifestyle risk score described above, including the digital device hours component normalized to a waking day. In these and other embodiments, the device use data 714 may accordingly serve as a source from which one or more components of the lifestyle data 704 may be derived or supplemented.

In some embodiments, another data domain of the previous patient profile data 702 may include genetic data 716. The genetic data 716 may represent genomic and molecular biomarker information corresponding to previous IOL patients. In some embodiments, the genetic data 716 may include data pertaining to genetic variants, polymorphisms, or biomarker profiles of previous IOL patients that may have influenced their respective IOL outcomes, healing responses, or post-operative satisfaction levels. For example, the genetic data 716 may include data indicating the presence or absence of genetic variants associated with inflammatory response pathways, wound healing characteristics, or susceptibility to dry eye conditions, each of which may have influenced the post-operative experience of the respective previous IOL patient. The genetic data 716 may further include biomarker data corresponding to previous IOL patients, such as proteomic or metabolomic markers that may serve as indicators of ocular surface health or tissue response characteristics relevant to IOL outcomes.

In some embodiments, the genetic data 716 may be sourced from genomic testing platforms or laboratory assay systems that may be employed to analyze biological samples obtained from previous IOL patients. Additionally or alternatively, the genetic data 716 may be sourced from existing genomic data records associated with previous IOL patients, such as records stored within EHR systems accessible to the data management module 34 of the controller C. In these and other embodiments, the genetic data 716 may be retrieved by the data management module 34 from electronic medical records of various clinical facilities at which previous IOL patients received care.

In some embodiments, another data domain of the previous patient profile data 702 may include outcome data 718. The outcome data 718 may represent real-world results experienced by previous IOL patients following their respective IOL implantation procedures. In some embodiments, the outcome data 718 may include post-operative measurements, patient-reported satisfaction responses, one or more answers to one or more post-operative patient reported visual disturbance questionnaires, and/or clinical assessments obtained from previous IOL patients following their respective IOL procedures. In these and other embodiments, the outcome data 718 may include data indicating the degree to which a previous IOL patient reported satisfaction with their post-operative visual experience, including whether their post-operative outcomes met, exceeded, or did not meet their pre-operative expectations. The outcome data 718 may further include data indicating the degree of spectacle independence achieved by a previous IOL patient following their IOL procedure, including whether the previous IOL patient achieved complete spectacle independence, partial spectacle dependence, or full spectacle dependence. The outcome data 718 may further include data indicating the presence or absence of adverse visual phenomena reported by previous IOL patients following their respective IOL procedures, such as dysphotopsia, halos, glare, dry eye symptoms, and posterior capsule opacification.

In some embodiments, the outcome data 718 may include quantitative visual performance measurements obtained from previous IOL patients post-operatively. For example, the outcome data 718 may include post-operative visual acuity measurements, including uncorrected distance visual acuity (UDVA), uncorrected near visual acuity (UNVA), and distance-corrected near visual acuity (DCNVA), each of which may be categorized according to a predefined outcome classification scheme such as Excellent, Good, Fair, or Poor. The outcome data 718 may further include contrast sensitivity measurements and other quantitative assessments of post-operative visual function obtained by the health care provider 16 during post-operative clinical examinations. In some embodiments, the outcome data 718 may include post-operative imaging data, such as the post-operative image 500 of FIG. 6B, obtained following the IOL implantation procedure of the respective previous IOL patient.

In these and other embodiments, the outcome data 718 may include patient satisfaction data collected through structured post-operative questionnaires administered to previous IOL patients at defined post-surgical intervals. For example, the outcome data 718 may include responses provided by previous IOL patients to satisfaction surveys delivered via the smart device 20, including through the electronic virtual assistant 22 or a mobile application associated therewith. The outcome data 718 may further include responses provided by previous IOL patients through a web-based portal or through a link-based access mechanism delivered via SMS text message or email. In these and other embodiments, the outcome data 718 may include an overall patient satisfaction score, a likelihood-to-recommend score, and an ease-of-adaptation score, each of which may be expressed on a predefined numerical scale.

In some embodiments, the previous patient profile data 702 may be organized into individual patient profile vectors. An individual patient profile vector may represent a single patient and may aggregate data from the different data domains into a unified, structured representation of that patient. For example, in some embodiments, the patient profile vector for a given patient may be expressed as: P=[L, E, C, I, D, S, G, PRO], where each component of the vector corresponds to a respective one of the eight data domains described above. For instance, the component “L” may correspond to the lifestyle data 704 of the respective patient; the component “E” may correspond to the education data 706 of the respective patient; the component “C” may correspond to the clinical data 708 of the respective patient; the component “I” may correspond to the diagnostic data 710 of the respective patient; the component “D” may correspond to the demographic data 712 of the respective patient the component “S” may correspond to the device use data 714; the component “G” may correspond to the genetic data 716 of the respective patient; and the component “PRO” may correspond to the outcome data 718 of the respective patient.

In some embodiments, the previous patient profile data 702 may accordingly be organized as a collection of individual patient profile vectors, wherein each patient profile vector in the collection corresponds to a respective one of the prior IOL patients and encodes data from each of the eight data domains for that specific patient.

In some embodiments, the previous patient profile data 702 may be used to train one or more AI models of the prediction module 750 to make outcome predictions. For example, in some embodiments, the AI models of the prediction module 750 may be trained using a supervised learning approach. In such embodiments, the multi-domains of the previous patient profile data 702, except for the outcome data 718, may serve as input features, and the outcome data 718 may serve as the corresponding target labels or ground truth values against which the AI models may be trained. For example, the AI models may be trained to learn associations between the multi-domain input features of the previous patient profile data 702 and the corresponding outcome labels contained within the outcome data 718.

In some embodiments, the training dataset may be assembled by pairing, for each prior IOL patient, the input domains of a patient profile vector (e.g., [L, E, C, I, D, S, G]) with the corresponding outcome data 718 (e.g., [PRO]) recorded for that patient following their respective IOL implantation procedure. The AI models of the prediction module 750 may be configured to learn a mapping function that transforms the input domains of the patient profile vector into a predicted outcome distribution.

In some embodiments, the training of the AI models of the prediction module 750 may incorporate a patient profile similarity formula for different profiles of the prior IOL patients. In such embodiments, the AI models may be trained to weight clinical and non-clinical similarity components according to their respective contributions to outcome prediction accuracy, as determined from the previous patient profile data 702. In some embodiments, the weights applied to the clinical similarity component and the non-clinical similarity component SNC may be learned during training by minimizing a cost function defined over the outcome data 718 of the prior IOL patients.

In some embodiments, the AI models of the prediction module 750 may be trained to generate probabilistic outcome forecasts across multiple outcome dimensions. For example, the AI models may be trained to predict a probability distribution over visual acuity achievement categories, a probability distribution over patient satisfaction scores, a probability distribution over spectacle independence classifications, and a probability distribution over adverse event occurrences, each derived from the corresponding outcome data 718 within the previous patient profile data 702. In these and other embodiments, an expected utility formula may be applied using the trained probability distributions to compute a Probability of Success score for different candidate IOL technologies.

The candidate profile data 722 may correspond to the candidate patient and may include data about the candidate patient corresponding to different data domains that may be useful in determining the outcome prediction 738 for the candidate patient. In some embodiments, the candidate profile data 722 may be similar or analogous to the previous patient profile data 702. For example, the candidate profile data 722 may include lifestyle data 724 that is similar or analogous to the lifestyle data 704; education data 726 that is similar or analogous to the education data 706; clinical data 728 that is similar or analogous to the clinical data 708; diagnostic data 730 that is similar or analogous to the diagnostic data 710; demographic data 732 that is similar or analogous to the demographic data 712; device use data 734 that is similar or analogous to the device use data 714; and/or genetic data 736 that is similar or analogous to the genetic data 716.

In some embodiments, the candidate profile data 722 may be organized into a candidate profile. In these and other embodiments, the candidate profile may be organized into a candidate profile vector similar to the patient profile vectors of the previous patient profile data. The difference may be that the candidate profile vector may not include any outcome data given. For example, the candidate profile vector may be expressed as: P_{Candidate}=[L, E, C, I, D, S, G].

The eligibility criteria 720 may represent a set of predefined conditions, thresholds, or rules that may be used to make a clinical determination as to whether a patient (e.g., the candidate patient) may be eligible for an IOL procedure. In some embodiments, the eligibility criteria 720 may serve as a gating mechanism that determines whether a given intraocular lens may be clinically suitable for a patient prior to further outcome analysis.

For example, the eligibility criteria 720 may include one or more mandatory clinical inclusion or exclusion conditions that may be evaluated on a per-technology basis for each intraocular lens. For example, the eligibility criteria 720 may include a glaucoma criterion, a retinal pathology criterion (such as macular degeneration, epiretinal membrane, or diabetic macular edema), a corneal irregularity criterion, a prior refractive surgery criterion (such as a history of LASIK or PRK), a dry eye severity criterion, and one or more lifestyle-informed criteria (such as night driving activity or dysphotopsia risk). In these and other embodiments, the eligibility criteria 720 may reflect both objective clinical contraindications and patient-specific compatibility considerations that may affect the suitability of certain IOL platforms.

As indicated above, the outcome prediction module 750 may be configured to generate the outcome prediction 738 for the candidate patient based on the previous patient profile data 702, the eligibility criteria 720, and/or the candidate profile data 722. For example, outcome prediction module 750 may be configured to compare the candidate profile data 722 against the eligibility criteria 720 to determine whether the candidate patient is clinically eligible for an IOL procedure.

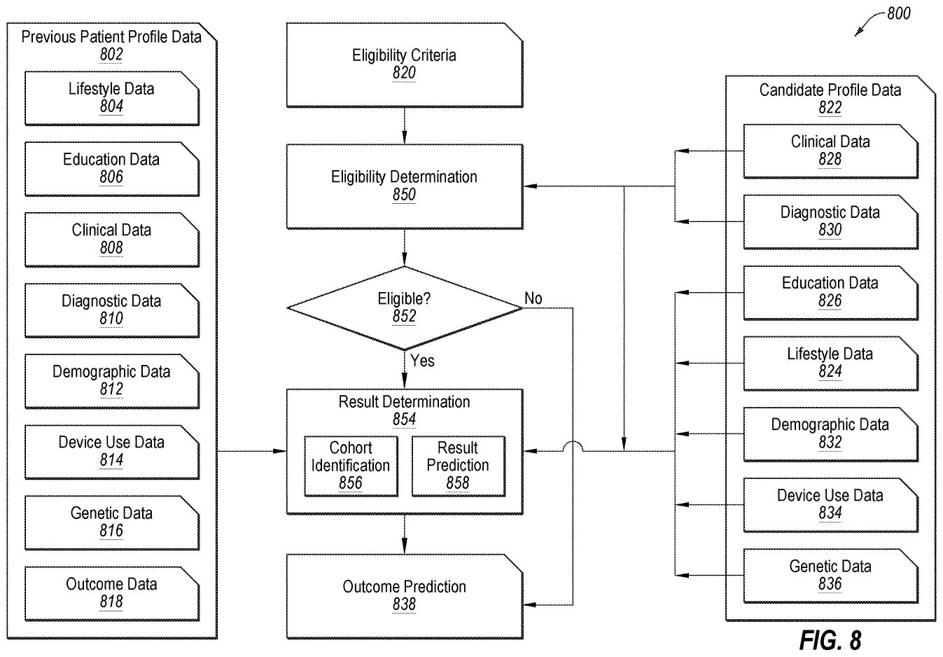

Additionally or alternatively, the outcome prediction module 750 may be configured to apply one or more AI models to compare the candidate profile data 722 against the previous patient profile data 702. For example, the outcome prediction module 750 may identify, based on the comparison, a cohort of previous IOL patients whose profiles resemble the profile of the candidate patient. Additionally or alternatively, the outcome prediction module 750 may be configured to use the outcome data 718 associated with the identified cohort to generate probabilistic outcome forecasts for the candidate patient across one or more outcome dimensions to determine the outcome prediction 738. In some embodiments, the outcome prediction module 750 may perform one or more operations of a process 800 described with respect to FIG. 8 to generate or determine the outcome prediction 738.

In some embodiments, the outcome prediction module 750 may include multiple AI models that may be used by the outcome prediction module 750. For example, in some embodiments, the AI models of the outcome prediction module 750 may include one or more of a Bayesian inference model, a deep multimodal learning model, a graph neural network, or an ensemble model. In these and other embodiments, different AI models may be selected for use by the outcome prediction module 750 based on different dataset characteristics of the previous patient profile data 702 and/or the candidate profile data 722. In these and other embodiments, the outcome prediction module 750 may be configured to identify the different dataset characteristics and/or the candidate profile data 722 and select which AI model to use. In these and other embodiments, the outcome prediction module 750 may be provided with an indication as to which AI model to use.

In some embodiments, dataset characteristics that may influence AI model selection may include the size of the available dataset, the dimensionality of the input feature space, the modality of the data, the degree of uncertainty present in the data, the presence or absence of labeled outcome data, the degree of class imbalance in the outcome data 718, the temporal structure of the data, the relational structure among patient profiles, the availability of imaging data, and/or the computational resources available for model training and inference.

For example, when the previous patient profile data 702 available for a particular predefined category is limited in volume, the prediction module 750 may be configured to apply a Bayesian inference model. Conversely, when the previous patient profile data 702 is large in volume and/or includes complex data (e.g., imaging data), the prediction module 750 may be configured to apply a deep multimodal learning model.

As another example, the relational structure among patient profiles may be another characteristic that influences AI model selection. For instance, when the previous patient profile data 702 exhibits a network-like relational structure, the prediction module 750 may be configured to apply a graph neural network.

Additionally or alternatively, the requirement for production-grade robustness and stability may be a further characteristic that influences AI model selection. For example, when the previous patient profile data 702 spans multiple predefined categories simultaneously—such as when the training dataset includes patients from multiple health care providers 16, multiple facilities, and/or multiple geographic regions—the prediction module 750 may be configured to apply an ensemble model. An ensemble model may be suited to heterogeneous, multi-source datasets because it may combine the outputs of multiple constituent models, each trained on a different subset or representation of the previous patient profile data 702, into a single aggregated prediction.

The outcome prediction 738 may encompass one or more predictions pertaining to the candidate patient's anticipated experience with one or more IOL technologies and/or procedures. In these and other embodiments, the outcome prediction 738 may include a determination of whether the candidate patient may be eligible for an IOL procedure at all. For example, based on the candidate profile data 722 and the one or more eligibility criteria 720, the outcome prediction 738 may indicate that the candidate patient may not be a suitable candidate for any IOL procedure, or alternatively, that the candidate patient may be a suitable candidate for one or more IOL procedures.

Additionally or alternatively, in some embodiments, the outcome prediction 738 may include predictions of success for different IOL technologies. within the group G of intraocular lenses. For example, referring to the group G of intraocular lenses, the outcome prediction 738 may include a predicted probability of success for each of the first intraocular lens L1, the second intraocular lens L2, and the third intraocular lens L3 (or any other IOLs). In these and other embodiments, the predicted probability of success for each IOL technology may be expressed as a Probability of Success score, which may be expressed as a percentage accompanied by a confidence interval that reflects the degree of uncertainty associated with the prediction. The outcome prediction 738 may further include probabilistic forecasts across multiple outcome dimensions for each IOL technology, including, for example, a predicted probability distribution over visual acuity achievement categories, a predicted probability distribution over patient satisfaction scores, a predicted probability distribution over spectacle independence classifications, and/or a predicted probability of adverse events or complications.

In these and other embodiments, the outcome prediction 738 may include a ranking of the IOL technologies. For example, the outcome prediction 738 may rank the first intraocular lens L1, the second intraocular lens L2, and the third intraocular lens L3 in descending order of their respective Probability of Success scores, such that the IOL technology with the highest predicted probability of success may appear at the top of the ranking. Additionally or alternatively, the ranking may be accompanied by the respective confidence intervals for each ranked IOL technology, enabling the health care provider 16 to assess the degree of certainty associated with each ranked position. In these and other embodiments, the outcome prediction 738 may further include a ranked comparison of the predicted outcome dimension forecasts across the ranked IOL technologies, such that the health care provider 16 may assess not only the overall Probability of Success ranking but also the relative predicted performance of each IOL technology across individual outcome dimensions such as patient satisfaction and/or spectacle independence.

Additionally or alternatively, the system 700 may include an IOL selection module 752 (“selection module 752”). The selection module 752 may be configured to generate an IOL selection 740 based on the outcome prediction 738. In some embodiments, the IOL selection 740 may represent a recommended intraocular lens selected for implantation in the candidate patient from one or more potential intraocular lens.

In some embodiments, the selection module 752 may be configured to generate the IOL selection 740 by applying a selection criterion to the outcome prediction 738. For example, the selection criterion may specify that the IOL selection 740 may correspond to the intraocular lens associated with the highest Probability of Success score in the outcome prediction 738. Additionally or alternatively, the selection criterion may incorporate one or more of the outcome dimension forecasts included in the outcome prediction 738. For example, the selection module 752 may be configured to weight the predicted probability distributions over patient satisfaction scores and spectacle independence classifications when generating the IOL selection 740, such that the IOL selection 740 may reflect not only the overall Probability of Success ranking but also the relative predicted performance of each intraocular lens across individual outcome dimensions.

In these and other embodiments, the selection module 752 may be configured to generate the IOL selection 740 as a weighted recommendation, similar to the weighted recommendations described above with respect to block 112 of FIG. 2. For example, the IOL selection 740 may assign a respective weighting factor to each intraocular lens in the group G based on the corresponding Probability of Success score in the outcome prediction 738, such that the intraocular lens with the highest Probability of Success score may receive the highest weighting factor. In these and other embodiments, the intraocular lens having the highest weighting factor may be selected by default as the IOL selection 740, subject to review and override by the health care provider 16.

Modifications, additions, or omissions may be made to FIG. 7 without departing from the scope of the present disclosure. For example, the system 700 may be configured with a greater or lesser number of data domains within the previous patient profile data 702 and the candidate profile data 722 than those illustrated in FIG. 7. In some embodiments, one or more of the data domains depicted in FIG. 7 may be omitted from the previous patient profile data 702 or the candidate profile data 722. In these and other embodiments, the absence or omission of such data may be treated as that area not being included in the analysis of the outcome prediction module 750, or may be treated as having an average score, a negative score, or a positive score. Additionally or alternatively, the absence or omission of such data may affect the weight given to a respective factor. Additionally or alternatively, in some embodiments, additional data domains beyond those illustrated in FIG. 7 may be incorporated into the previous patient profile data 702 and the candidate profile data 722.

Further, in some embodiments, the outcome prediction module 750 and the IOL selection module 752 may be combined into a single processing module rather than being represented as separate modules as illustrated in FIG. 7. Additionally or alternatively, the outcome prediction module 750 and/or the IOL selection module 752 may be subdivided into multiple sub-modules. Additionally, in some embodiments, the eligibility criteria 720 may be incorporated directly into the outcome prediction module 750 rather than being represented as a separate input block as shown in FIG. 7. In these and other embodiments, the outcome prediction 738 and the IOL selection 740 may be combined into a single output rather than being represented as separate outputs as illustrated in FIG. 7. The data flow arrows depicted in FIG. 7 may also vary in number, direction, or connectivity in other embodiments without departing from the scope of the present disclosure.

FIG. 8 illustrates an example IOL outcome prediction process 800 (“process 800”) that may be used to generate an outcome prediction 838 with respect to an IOL procedure for a candidate patient for the IOL procedure. The process 800 may be performed by any suitable system, apparatus, or device. For example, in some embodiments, one or more of the operations of the process 800 may be performed by the outcome prediction module 750 of FIG. 7 and/or by the controller C of FIG. 1.

The process 800 may be configured to use eligibility criteria 820, previous patient profile data 802, and candidate profile data 822 in making the outcome prediction 838. The eligibility criteria 820 may be similar or analogous to the eligibility criteria 720 of FIG. 7. The previous patient profile data 802 may include lifestyle data 804, education data 806, clinical data 808, diagnostic data 810, demographic data 812, device use data 814, genetic data 816, and outcome data 818, each of which may be similar or analogous to the corresponding data types of the previous patient profile data 702 of FIG. 7. The candidate profile data 822 may include lifestyle data 824, education data 826, clinical data 828, diagnostic data 830, demographic data 832, device use data 834, and genetic data 836, each of which may be similar or analogous to the corresponding data types of the candidate profile data 722 of FIG. 7. The outcome prediction 838 may be similar or analogous to the outcome prediction 738 of FIG. 7.

In some embodiments, the process 800 may include an eligibility determination operation 850. The eligibility determination operation 850 may be configured to evaluate the candidate profile data 822 against the eligibility criteria 820 to determine whether the candidate patient may be clinically eligible for an IOL procedure. For example, in some embodiments, the eligibility determination operation 850 may include extracting the clinical data 828 and the diagnostic data 830 from the candidate profile data 822. The eligibility determination operation 850 may then include evaluating the extracted clinical data 828 and diagnostic data 830 against the eligibility criteria 820. For example, the eligibility determination operation 850 may evaluate whether the candidate patient exhibits a condition corresponding to a glaucoma criterion, a retinal pathology criterion, a corneal irregularity criterion, a prior refractive surgery criterion, a dry eye severity criterion, or one or more lifestyle-informed criteria included within the eligibility criteria 820 that may affect whether the candidate patient is eligible for an IOL procedure.

In some embodiments, such evaluation may be based on a per-technology basis of different IOL technologies. For example, the eligibility determination operation 850 may include evaluating the candidate profile data 822 against each eligibility criterion independently for each IOL technology within the group G of intraocular lenses, such that a separate eligibility determination may be generated for each of the first intraocular lens L1, the second intraocular lens L2, and the third intraocular lens L3.

Following the eligibility determination operation 850, the process 800 may proceed to a decision block 852. The decision block 852 may represent a determination of whether the candidate patient may be eligible for an IOL procedure with respect to a given IOL technology. In some embodiments, the decision block 852 may assign a binary pass or fail determination to each IOL technology evaluated during the eligibility determination operation 850. Where the candidate patient may not satisfy the eligibility criteria 820 for a given IOL technology, the decision block 852 may follow a “No” path, indicating that the candidate patient may not be eligible for that IOL technology. In such cases, the ineligibility determination itself may be used as the outcome prediction 838 for that particular IOL technology.

For example, the outcome prediction 838 for an IOL technology that fails the eligibility determination may indicate that the candidate patient may not be a suitable candidate for that technology, and may further include the specific eligibility criterion that was not satisfied, such as the presence of significant retinal pathology or a corneal irregularity that may be incompatible with the IOL technology under evaluation. In this manner, the ineligibility determination may serve as a clinically grounded outcome prediction that communicates to the health care provider 16 the specific clinical basis for the exclusion of a given IOL technology from further consideration.

Where the candidate patient may satisfy the eligibility criteria 820 for a given IOL technology, the decision block 852 may follow a “Yes” path, indicating that the candidate patient may be eligible for that IOL technology. In such cases, the process 800 may proceed to a result determination operation 854.

The result determination operation 854 may include determining a probability of success for the eligible IOL technology with respect to the candidate patient. In some embodiments, the result determination operation 854 may draw upon both the candidate profile data 822 and the previous patient profile data 802 to generate a probabilistic outcome forecast for the candidate patient. In some embodiments, the result determination operation 854 may include a cohort identification operation 856 and a result prediction operation 858.

The cohort identification operation 856 may include identifying, from among the previous patient profile data 802, a cohort of prior IOL patients whose profiles may resemble the profile of the candidate patient included in the candidate profile data 822. In some embodiments, the cohort identification operation 856 may include determining a patient profile similarity score between the candidate profile data 822 and each individual patient profile within the previous patient profile data 802.

For example, in some embodiments, the patient profile similarity score between the candidate patient (“P₁”) and a previous IOL patient (“P₂”) (“S(P₁, P₂)”) may be computed according to a patient profile similarity formula as follows:

$$S ( {P}_{1} , {P}_{2} ) = {W}_{C} * {S}_{C} + {W}_{N C} * {S}_{N C}$$

In the above expression, S_{C} relates to a clinical similarity component, which may be derived from a comparison of the clinical data 828 and diagnostic data 830 of the candidate profile data 822 against the corresponding clinical data 808 and diagnostic data 810 of the corresponding prior patient profile within the previous patient profile data 802. Further, in the above expression, S_{NC} relates to a non-clinical similarity component, which may be derived from a weighted combination of comparisons across the lifestyle data 824, education data 826, demographic data 832, and device use data 834 of the candidate profile data 822 against the corresponding lifestyle data 804, education data 806, demographic data 812, and device use data 814 of the corresponding prior patient profile within the previous patient profile data 802.

In some embodiments, the cohort identification operation 856 may select the AI methodology appropriate to the characteristics of the previous patient profile data 802 and the candidate profile data 822. For example, where the previous patient profile data 802 may be limited in volume, the cohort identification operation 856 may apply a Bayesian inference model to compute similarity scores and identify the cohort. Where the previous patient profile data 802 may be large in volume and may include complex imaging data, the cohort identification operation 856 may apply a deep multimodal learning model. Where the previous patient profile data 802 may exhibit a network-like relational structure among patient profiles, the cohort identification operation 856 may apply a graph neural network. Where the previous patient profile data 802 may span multiple predefined categories simultaneously, the cohort identification operation 856 may apply an ensemble model. The cohort identification operation 856 may output an identified cohort of prior IOL patients, together with the associated outcome data 818 corresponding to those cohort members, for use by the result prediction operation 858.

In some embodiments, the result prediction operation 858 may include determining a probability of success for the eligible IOL technology based on the outcome data 818 associated with the identified cohort. In these and other embodiments, the result prediction operation 858 may generate probabilistic outcome forecasts across multiple outcome dimensions for the corresponding IOL technology. For example, the result prediction operation 858 may include generating a predicted probability distribution over visual acuity achievement categories, including uncorrected distance visual acuity, uncorrected near visual acuity, and distance-corrected near visual acuity, each of which may be categorized according to a predefined outcome classification scheme such as Excellent, Good, Fair, or Poor, derived from the outcome data 818 of the identified cohort.

Additionally or alternatively, the result prediction operation 858 may include generating a predicted probability distribution over patient satisfaction scores, including an overall satisfaction score, an ease of adaptation score, and/or a likelihood-to-recommend score, each derived from the corresponding outcome data 818 of the identified cohort.

In these and other embodiments, the result prediction operation 858 may include generating a predicted probability distribution over spectacle independence classifications, including complete spectacle independence, partial spectacle dependence, and full spectacle dependence, derived from the outcome data 818 of the identified cohort. Additionally or alternatively, the result prediction operation 858 may include generating a predicted probability of adverse events or complications, including dysphotopsia, dry eye symptoms, and posterior capsule opacification, derived from the outcome data 818 of the identified cohort. In some embodiments, the result prediction operation 858 may apply an expected utility formula to the predicted probability distributions to compute an overall probability of success score for the eligible IOL technology.

For example, in some embodiments, the expected utility formula may be expressed as follows:

$$EU ( Tech ) = \sum P ( {Outcome}_{i} \vert P , Tech ) * Utility ( {Outcome}_{i} )$$

In the above expression “P(Outcomeᵢ|P, Tech)” may represent the predicted probability of a given outcome for the candidate patient given the candidate profile data 822 and the eligible IOL technology, and “Utility(Outcomeᵢ)” may represent a predefined or patient-customized utility weighting for each outcome category.

In these and other embodiments, the result prediction operation 858 may further include determining a confidence interval for the probability of success score, reflecting the degree of uncertainty associated with the prediction—e.g., as determined from the size and composition of the identified cohort. The output of the result prediction operation 858 may contribute to the outcome prediction 838, which may include the probability of success score, the associated confidence interval, and/or the outcome dimension forecasts for the eligible IOL technology.

Modifications, additions, or omissions may be made to the process 800 without departing from the scope of the present disclosure. For example, in some embodiments, the process 800 need not be applied in the specific order recited herein and some blocks may be omitted. Modifications, additions, or omissions may be made to the process 800 without departing from the scope of the present disclosure.

For example, in some embodiments, the process 800 may be modified such that the eligibility determination 850 and the result determination 854 are combined into a single processing stage rather than being represented as sequential stages as illustrated in FIG. 8. Additionally or alternatively, the eligibility criteria 820 may be incorporated directly into the result determination 854 rather than being applied as a separate upstream gating step. In some embodiments, the decision diamond 852 may be omitted such that the process 800 proceeds directly from the eligibility determination 850 to the result determination 854 without a discrete binary pass or fail determination. In such embodiments, the eligibility assessment may instead be expressed as a continuous eligibility score that may be incorporated as a weighted input into the result determination 854.

Further, in some embodiments, the cohort identification 856 and the result prediction 858 within the result determination 854 may be performed iteratively rather than in a single pass. For example, the result prediction 858 may generate a preliminary outcome prediction 838, which may then be used to refine the cohort identification 856 in a subsequent iteration, with the refined cohort then informing an updated result prediction 858. Additionally or alternatively, the cohort identification 856 may be omitted in some embodiments, such that the result prediction 858 may operate directly on the previous patient profile data 802 and the candidate profile data 822 without an intermediate cohort identification step. For instance, the cohort identification 856 may be implicit in some AI models that were trained on previous patient profile data in that different weights internal to the AI models may be based on different input data modalities. As such, the outcome prediction 838 obtained by supplying the candidate profile data 822 to the AI models may be based on application of the different weights to the input data modalities of the candidate profile data 822.

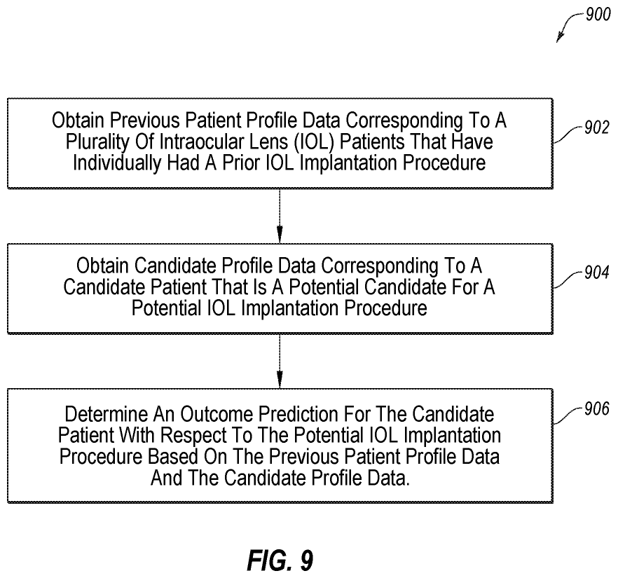

FIG. 9 illustrates a flowchart of an example method 900 for determining an outcome prediction of an IOL procedure. The method 900 may be arranged in accordance with at least one embodiment described in the present disclosure. One or more operations of the method 900 may be performed, in some embodiments, by a device or system, such as the controller C of FIG. 1 and/or the system 700 of FIG. 7, or another device or combination of devices. In these and other embodiments, the method 900 may be performed based on the execution of instructions stored on one or more non-transitory computer-readable media. Further, in some embodiments, the method 900 may include one or more of the operations of the process 800. Although illustrated as discrete blocks, various blocks may be divided into additional blocks, combined into fewer blocks, or eliminated, depending on the desired implementation.

The method 900 may include block 902. Block 902 may include obtaining previous patient profile data corresponding to a plurality of IOL patients that may have individually had a prior IOL implantation procedure. The previous patient profile data may correspond to the previous patient profile data 702 of FIG. 7.

At block 904, the method 900 may include obtaining candidate profile data corresponding to a candidate patient that may be a potential candidate for a potential IOL implantation procedure. The candidate profile data may correspond to the candidate profile data 722 of FIG. 7.

At block 906, the method 900 may include determining an outcome prediction for the candidate patient with respect to the potential IOL implantation procedure based on the previous patient profile data and the candidate profile data. The outcome prediction may correspond to the outcome prediction 738 of FIG. 7. Determining the outcome prediction may include performing one or more operations of the process 800 in some embodiments.

It is understood that, for this and other processes, operations, and methods disclosed herein, the functions and/or operations performed may be implemented in differing order. Furthermore, the outlined functions and operations are only provided as examples, and some of the functions and operations may be optional, combined into fewer functions and operations, or expanded into additional functions and operations without detracting from the essence of the disclosed embodiments.

FIG. 10 illustrates a flowchart of an example method 1000 for curating education materials for a candidate patient of an IOL procedure. The method 1000 may be arranged in accordance with at least one embodiment described in the present disclosure. One or more operations of the method 1000 may be performed, in some embodiments, by a device or system, such as the controller C of FIG. 1 and/or the system 700 of FIG. 7, or another device or combination of devices.

The method 1000 may include block 1002. At block 1002, the method 1000 may include obtaining candidate profile data corresponding to a candidate patient that may be a potential candidate for a potential IOL implantation procedure. The candidate profile data may correspond to the candidate profile data 722 of FIG. 7.

The method 1000 may include block 1004. At block 1004, the method 1000 may include determining an education level of the candidate patient with respect to IOL implantation. In some embodiments, the education level determination may be made based on an artificial intelligence (AI) analysis of the candidate profile data. In these and other embodiments, the education level may correspond to the Education Understanding Score described above with respect to the education data 706 of FIG. 7.

The method 1000 may include block 1006. At block 1006, the method 1000 may include curating education materials for the candidate patient based on the determined education level and based on the candidate profile data 722. In some embodiments, the curated education materials may be assembled from a repository of structured educational content items organized by clinical topic, IOL type, patient condition, and other relevant attributes. The curated education materials may include text, video, graphics, and interactive media items that are selected based on the determined education level and the candidate profile data.

In some embodiments, the curating at block 1006 may include selecting educational content items that are deemed to be clinically relevant and personally applicable to the candidate patient's specific profile, while excluding educational content items that may be irrelevant, confusing, or potentially misleading for the candidate patient. For example, where the candidate profile data indicates the presence of a condition such as glaucoma, the curating at block 1006 may include excluding educational content pertaining to presbyopia-correcting IOLs from the curated education materials. Additionally or alternatively, where the candidate profile data indicates that the candidate patient has previously worn multifocal contact lenses, the curating at block 1006 may include selecting educational content pertaining to multifocal IOLs and astigmatism correction for inclusion in the curated education materials.

In some embodiments, the curating at block 1006 may include adjusting the complexity and specificity of the educational content items based on the determined education level of the candidate patient. For example, where the determined education level indicates a lower Education Understanding Score, the curating at block 1006 may include selecting educational content items that employ simplified language and foundational explanations of IOL technology. Conversely, where the determined education level indicates a higher Education Understanding Score, the curating at block 1006 may include selecting educational content items that employ more technically detailed descriptions of IOL optical characteristics and expected outcomes.

In some embodiments, the curating at block 1006 may include curating the education materials based on the lifestyle data 724 of the candidate patient. For example, the lifestyle data 724 may indicate that the candidate patient engages in recreational activities requiring near vision, such as reading or detailed close-range work, in which case the curating at block 1006 may include selecting educational content pertaining to IOL options that may address near vision demands. Additionally or alternatively, the lifestyle data 724 may indicate that the candidate patient engages in activities requiring distance vision, such as outdoor sports or driving, in which case the curating at block 1006 may include selecting educational content pertaining to IOL options that may address distance vision demands.

In some embodiments, the curating at block 1006 may include curating the education materials based on demographic data included in the candidate patient data, which may include age, general education level, and/or socioeconomic status. For example, the controller C may be configured to select educational content items that correspond to the candidate patient's education level or age, such that the curated materials may be presented at an appropriate level of explanation.

For example, where the demographic data indicates a younger age or higher education level, the curating may include selecting content employing more technical language and assuming greater familiarity with medical and optical concepts. Conversely, where the demographic data indicates an older age or lower education level, the curating may include selecting content employing simplified language, foundational explanations, and accessible analogies.

In some embodiments, the repository may include multiple versions of content items for the same clinical topic, each written at a different complexity or reading grade level. The curating at block 1006 may include selecting the version corresponding to the education level or age indicated by the demographic data in some embodiments.

Additionally or alternatively, in some embodiments, the curating may further include adjusting the format and modality of the content items based on the demographic data. For example, for older patients, the curating may include selecting content available in larger fonts, higher contrast visuals, or audio-narrated video formats. For younger or more educated patients, the curating may include selecting interactive digital or detailed written formats.

As another example, in some embodiments, where the demographic data includes socioeconomic status information, the controller C may be configured to select content items addressing the financial considerations of different IOL options in a manner appropriate to the candidate patient's socioeconomic context.

In some embodiments, the curating may include combining the education level determination from block 1004 with the demographic data to generate a composite patient comprehension profile. For example, the controller C may be configured to select content items based on the composite profile, reflecting both the Education Understanding Score and the demographic data. Where these indicate different comprehension levels, the controller C may apply a predefined reconciliation rule, such as selecting the lower comprehension level to ensure accessibility.

In some embodiments, the curating at block 1006 may include curating the education materials based on device use data included in the candidate patient profile. For example, the device use data 734 may indicate the viewing distance D at which the candidate patient holds a smart device relative to their eye, which may be indicative of the near vision demands of the candidate patient, as described above with respect to block 104 of FIG. 2. The curating at block 1006 may accordingly include selecting educational content pertaining to IOL options that may address the near vision demands indicated by the viewing distance D.

Additionally or alternatively, in some embodiments, the curating at block 1006 may include curating the education materials based on clinical data and/or diagnostic data included in the candidate profile data. For example, where the diagnostic data includes pre-operative imaging data such as the pre-operative image 400 of the eye E shown in FIG. 6A, the curating at block 1006 may include selecting educational content pertaining to IOL options that may be compatible with the anatomical parameters of the candidate patient's eye. Additionally or alternatively, where the clinical data indicates a prior refractive surgical history such as LASIK or PRK, the curating at block 1006 may include selecting educational content that addresses the implications of prior refractive surgery for IOL selection and expected outcomes.

In some embodiments, the clinical data may include pharmacy records (e.g., obtained from electronic health record systems). The pharmacy records may indicate medications that have been prescribed to the candidate patient, including medications for the treatment of ocular conditions such as glaucoma. For example, the pharmacy records may indicate that the candidate patient has been prescribed glaucoma medications such as prostaglandin analogs, beta-blockers, alpha agonists, or carbonic anhydrase inhibitors. The presence of such medications in the pharmacy records may serve as an indicator that the candidate patient has glaucoma or ocular hypertension, which may be relevant to the curating of IOL education materials. In these and other embodiments, where the pharmacy records indicate the presence of glaucoma medications, the curating at block 1006 may include excluding educational content pertaining to presbyopia-correcting IOLs from the curated education materials, as described above. Additionally or alternatively, the curating at block 1006 may include selecting educational content pertaining to simultaneous glaucoma surgical procedures that may be performed concurrently with cataract surgery, as described above. In these and other embodiments, the pharmacy records may be used to identify ocular comorbidities that may not have been explicitly documented in other portions of the electronic health record, thereby enabling more accurate curation of education materials that are clinically relevant to the candidate patient's specific condition.

In these and other embodiments, the curating at block 1006 may include curating the education materials based on an outcome prediction determined for the candidate patient, such as the outcome prediction 738 described above with respect to FIG. 7. For example, where the outcome prediction indicates a higher probability of success for a particular IOL technology, the curating at block 1006 may include selecting educational content pertaining to that IOL technology for inclusion in the curated education materials. In some embodiments, the curated education materials may include predicted satisfaction information derived from the outcome data of prior IOL patients with similar profiles to the candidate patient, as described above with respect to the previous patient profile data 702 of FIG. 7.

Additionally or alternatively, in some embodiments, the curating at block 1006 may include updating the curated education materials based on new information obtained at a later stage of the candidate patient's pre-operative journey. For example, where updated diagnostic data reveals new or previously undetected clinical findings, such as a corneal irregularity or unrecognized glaucoma, the curating at block 1006 may include updating the curated education materials to reflect the revised IOL recommendation that may result from those findings. Additionally or alternatively, the updating of the curated education materials may be triggered when a subsequent AI analysis of the additional candidate profile data generates an IOL recommendation that differs from an initial IOL recommendation generated based on the candidate profile data obtained prior to the pre-operative examination. In such embodiments, the updated curated education materials may be delivered to the candidate patient with an indication that the content reflects updated clinical findings, helping ensure that the candidate patient's understanding and expectations may remain aligned with the most current and complete clinical data available.

In some embodiments, the curated education materials may be delivered to the candidate patient via a smart device (e.g., the smart device 20) and/or through an electronic virtual assistant (e.g., the electronic virtual assistant 22) or a mobile application associated therewith. Additionally or alternatively, the curated education materials may be delivered via a web-based portal or via a link-based access mechanism delivered through an SMS text message or email, such as described above with respect to the smart device 20 of FIG. 1. In some embodiments, the delivery modality may be selected based on the device use data of the candidate patient, such that the curated education materials may be delivered through the access modality most consistent with the candidate patient's observed behavioral patterns with the smart device.

In some embodiments, the curated education materials may be delivered to the candidate patient via a personalized link. The personalized link may direct the candidate patient to a web-based interface through which the curated education materials may be accessed. In some embodiments, the personalized link may be associated with a unique access token that may be generated for the specific candidate patient. The unique access token may be embedded within the personalized link such that the link may be associated with the candidate profile data corresponding to that specific candidate patient.

In these and other embodiments, the personalized link may be configured to present the candidate patient with an identity verification prompt upon selection of the link. For example, the candidate patient may be prompted to enter a verification credential, such as a date of birth, before the curated education materials may be accessed. The identity verification prompt may serve to validate that the personalized link may have been received by the intended candidate patient. Upon successful entry of the verification credential, the candidate patient may be granted access to the curated education materials assembled for their specific candidate profile.

In some embodiments, the curated materials may be delivered to the candidate patient at a defined point along the pre-operative patient journey. For example, the personalized link may be delivered to the candidate patient prior to the candidate patient's scheduled consultation with the health care provider 16 of FIG. 1, such that the candidate patient may review the curated education materials before arriving at the clinical facility. In some embodiments, the curated materials may be delivered to the candidate patient at multiple points along the pre-operative journey. For example, first curated materials may be delivered to the candidate patient following the collection of initial candidate profile data at block 1002 of FIG. 10, and second curated materials may be delivered to the candidate patient following a determination of an updated education level at block 1004.

In some embodiments, the web-based interface (e.g., accessible via the personalized link) may be configured to record engagement data corresponding to the candidate patient's interaction with the curated education materials. For example, the web-based interface may record the duration of the candidate patient's engagement with each educational content item, the number of content items reviewed, and whether the candidate patient may have sought additional information by posing follow-up questions (e.g., to the electronic virtual assistant 22 of FIG. 1). In some embodiments, the engagement data recorded through the personalized link may be used to update the education data within the candidate profile data. In some embodiments, the engagement data may accordingly serve as an additional input to the education level determination at block 1004 of FIG. 10.

It is understood that, for this and other processes, operations, and methods disclosed herein, the functions and/or operations performed may be implemented in differing order. Furthermore, the outlined functions and operations are only provided as examples, and some of the functions and operations may be optional, combined into fewer functions and operations, or expanded into additional functions and operations without detracting from the essence of the disclosed embodiments.

The detailed description and the drawings or FIGS. are supportive and descriptive of the disclosure, but the scope of the disclosure is defined solely by the claims. While some of the best modes and other embodiments for carrying out the claimed disclosure have been described in detail, various alternative designs and embodiments exist for practicing the disclosure defined in the appended claims. Furthermore, the embodiments shown in the drawings or the characteristics of various embodiments mentioned in the present description are not necessarily to be understood as embodiments independent of each other. Rather, it is possible that each of the characteristics described in one of the examples of an embodiment can be combined with one or a plurality of other desired characteristics from other embodiments, resulting in other embodiments not described in words or by reference to the drawings. Accordingly, such other embodiments fall within the framework of the scope of the appended claims.

The subject technology of the present disclosure is illustrated, for example, according to various aspects described below. Various examples of aspects of the present disclosure are described as numbered examples (1, 2, 3, etc.) for convenience. These are provided as examples and do not limit the present disclosure. The aspects of the various implementations described herein may be omitted, substituted for aspects of other implementations, or combined with aspects of other implementations unless context dictates otherwise. For example, one or more aspects of example 1 below may be omitted, substituted for one or more aspects of another example (e.g., example 2) or examples, or combined with aspects of another example The following is a non-limiting summary of some example implementations presented herein.

Example 1.A system for selecting an intraocular lens for implantation into an eye of a patient, the system comprising:
a controller having a processor and tangible, non-transitory memory on which instructions are recorded;
wherein the controller is configured to receive data from a smart device operable by the patient, the data received from the smart device indicating at least one input factor related to the patient;
wherein the controller is configured to select, based on the at least one input factor, a chosen machine learning model from a plurality of trained models adaptively trained with a respective training dataset drawn from at least one predefined category, the controller being configured to selectively execute the chosen machine learning model from the plurality of trained models; and
wherein the controller is configured to generate an automated recommendation for the intraocular lens from a group of intraocular lenses based in part on a plurality of input factors related to the patient, including at least one input factor as indicated by the data received from the smart device, and an output generated by the chosen machine learning model.

Example 2.The system of Example 1, wherein the plurality of input factors include the data received from the smart device, the data including usage details of the patient on the smart device, including a viewing distance between the patient and the smart device.

Example 3.The system of Example 2, wherein:
the smart device from which the data is received includes at least one of a mobile phone, smart watch, and tablet; and
the data received from the smart device includes visual performance measurements of the patient made with the smart device.

Example 4.The system of any of Examples 1-3, wherein the at least one predefined category includes a specific health care provider such that the respective training dataset is drawn exclusively from patient data of the specific health care provider.

Example 5.The system of any of Examples 1-4, wherein the at least one predefined category includes a specific facility such that the respective training dataset is drawn exclusively from patient data associated with the specific facility.

Example 6.The system of any of Examples 1-5, wherein the at least one predefined category includes a specific region such that the respective training dataset is drawn exclusively from patient data from the specific region.

Example 7.The system of any of Examples 1-6, wherein the at least one predefined category includes a specific demographic, including at least one of age, gender, ethnicity, patient motivation and socioeconomic status.

Example 8.The system of any of Examples 1-7, wherein:
the plurality of input factors include questions of the patient presented to an electronic virtual assistant; and
the electronic virtual assistant is adapted to answer the questions posed by the patient.

Example 9.The system of any of Examples 1-8, wherein the controller is configured to generate a respective weighted recommendation for each member of the group of intraocular lenses and the chosen machine learning model on generative artificial intelligence.

Example 10. The system of Example 9, wherein the group of intraocular lenses includes:
a first intraocular lens; and
a second intraocular lens adapted to provide improved distance vision and improved intermediate vision relative to the first intraocular lens.

Example 11. The system of any of Examples 1-10, wherein the plurality of input factors includes preoperative physical data from at least one imaging device, the controller being adapted to receive the preoperative physical data from the at least one imaging device, the at least one imaging device including at least one of a biometer, an OCT device, or a camera system.

Example 12. The system of any of Examples 1-11, wherein the controller is adapted to select the intraocular lens based in part on at least one comorbidity rule-out marker generated by the controller, including a cornea irregularity marker for the patient.

Example 13. The system of any of Examples 1-12, wherein the controller is adapted to select the intraocular lens based in part on at least one comorbidity rule-out marker generated by the controller, including an eye dryness marker for the patient.

Example 14. The system of any of Examples 1-13, wherein the controller is adapted to select the intraocular lens based in part on at least one comorbidity rule-out marker generated by the controller, including a retinal surface irregularity marker for the patient.

Example 15. An apparatus for selecting an intraocular lens for implantation in an eye of a patient, comprising:
a controller having a processor and tangible, non-transitory memory on which instructions are recorded;
wherein the controller is configured to receive data from a smart device operable by the patient, the data received from the smart device indicating at least one input factor related to the patient;
wherein the controller is configured to select, based on at least one input factor, a chosen machine learning model from a plurality of trained models, the controller being configured to selectively execute the chosen machine learning from the plurality of trained models;
wherein the plurality of trained models are adaptively trained with a respective training dataset drawn from at least one predefined category, the at least one predefined category including a specific health care provider such that the respective training dataset is drawn exclusively from respective patients of the specific health care provider;
wherein the controller is configured to generate an automated recommendation for the intraocular lens from a group of intraocular lenses based in part on a plurality of input factors related to the patient, including at least one input factor as indicated by the data received from the smart device, and an output generated by the chosen machine learning model; and
wherein the plurality of input factors include usage details of the patient on the smart device, including a viewing distance of the smart device from the eye of the patient.

Example 16. The apparatus of Example 15, wherein the at least one predefined category further includes a specific demographic, including at least one of age, gender, ethnicity, patient motivation and socioeconomic status.

Example 17. The apparatus of Example 16, wherein the plurality of input factors include the data received from the smart device, the data including queries generated by the patient and presented to the smart device.

Example 18. The apparatus of Example 16 or Example 17, wherein the controller is adapted to select the intraocular lens based in part on at least one comorbidity rule-out marker generated by the controller, the at least one comorbidity rule-out marker including at least one of a cornea irregularity marker, an optical quality marker, an eye dryness marker, or a retinal surface irregularity marker for the patient.

Example 19. The apparatus of any of Examples 15-18, wherein:
the controller is configured to generate a respective weighted recommendation for each member of the group of intraocular lenses; and
the group of intraocular lenses includes:
   a first intraocular lens; and
   a second intraocular lens adapted to provide improved distance vision and improved intermediate vision relative to the first intraocular lens.

Example 20. The apparatus of any of Examples 15-19, wherein the plurality of input factors include preoperative physical data from at least one imaging device, the controller being adapted to receive the preoperative physical data from the at least one imaging device, the at least one imaging device including at least one of a biometer, an OCT device, or a camera system.

Example 21. A method comprising any of the operations performed by the system of Examples 1-14 or the apparatus of Examples 15-20.

Example 22. A method comprising:
obtaining previous patient profile data corresponding to a plurality of intraocular lens (IOL) patients that have individually had a prior IOL implantation procedure;
obtaining candidate profile data corresponding to a candidate patient that is a potential candidate for a potential IOL implantation procedure; and
determining an outcome prediction for the candidate patient with respect to the potential IOL implantation procedure based on the previous patient profile data and the candidate profile data.

Example 23. The method of Example 22, wherein the previous patient profile data includes one or more of:
lifestyle data about the IOL patients;
education data corresponding to IOL implantation education received by the IOL patients prior to their respective prior IOL implantation procedures;
clinical data about the IOL patients;
diagnostic data about the IOL patients;
demographic data about the IOL patients;
electronic device use data corresponding to the IOL patients;
genetic data about the IOL patients; or
outcome data corresponding to outcomes of the prior IOL implantation procedures.

Example 24. The method of Example 22 or Example 23, wherein the candidate profile data includes one or more of:
lifestyle data about the candidate patient;
education data corresponding to IOL implantation education received by the candidate patient;
clinical data about the candidate patient;
diagnostic data about the candidate patient;
demographic data about the candidate patient;
electronic device use data corresponding to the candidate patient; or
genetic data about the candidate patient.

Example 25. The method of any of Examples 22-24, wherein the determining the outcome prediction is based on outcome data corresponding to the IOL patients of a same cohort as the candidate patient.

Example 26. The method of any of Examples 22-25, further comprising recommending a specific IOL for the candidate patient based on the outcome prediction.

Example 27. The method of any of Examples 22-26, wherein the outcome prediction includes a plurality of individual outcome predictions that individually predict outcomes related to different IOLs.

Example 28. The method of any of Examples 22-27, wherein the outcome prediction is determined using an artificial intelligence (AI) model that is trained using the previous patient profile data.

Example 29. The method of Example 28, further comprising selecting which AI model to use based on dataset characteristics of the previous patient profile data.

Example 30. The method of any of Examples 22-29, further comprising:
determining, using an electronic virtual assistant EVA and based on the candidate profile data, an education level of the candidate patient with respect to IOL implantation;
identifying, by the EVA, IOL education materials based on the education level; and
providing, by the EVA, the IOL education materials to the candidate patient.

Example 31. The method of Example 30, wherein the identifying the IOL education materials comprises filtering a library of available IOL education materials to exclude materials that are determined to not be relevant to the candidate patient based on the candidate profile data.

Example 32. The method of Example 30 or Example 31, wherein the identifying the IOL education materials comprises selecting materials that address specific ocular conditions identified in the candidate profile data.

Example 33. The method of any of Examples 30-32, further comprising updating the IOL education materials provided to the candidate patient based on additional candidate profile data obtained after an initial provision of the IOL education materials.

Example 34. The method of Example 33, wherein the additional candidate profile data includes clinical measurements obtained during a pre-operative examination.

Example 35. The method of any of Examples 30-34, wherein the identifying the IOL education materials is based on lifestyle data included in the candidate profile data indicating visual usage patterns of the candidate patient.

Example 36. The method of Example 35, wherein the lifestyle data includes at least one of reading distance, screen time, driving habits, or activity levels of the candidate patient.

Example 37. The method of any of Examples 30-36, wherein the identifying the IOL education materials comprises including education materials related to astigmatism correction when the candidate profile data indicates the candidate patient wears multifocal contact lenses.

Example 38. The method of any of Examples 30-37, wherein the determining the education level comprises analyzing questions posed by the candidate patient to the EVA.

Example 39. The method of any of Examples 30-38, further comprising providing updated IOL education materials to the candidate patient when pre-operative diagnostic data indicates a change in IOL recommendation compared to an initial recommendation.

Example 40. The method of any of Examples 30-39, wherein the identifying the IOL education materials is based on outcome data included in the previous patient profile data corresponding to IOL patients having similar characteristics to the candidate patient.

Example 41. A computing system configured to perform the method of any of Examples 22-40.

Example 42. A computer-readable media having instructions stored thereon that, in response to being executed by one or more processors causes a system to perform the method of any of Examples 22-40.

Example 43. A method comprising:
obtaining candidate profile data corresponding to a candidate patient that is a potential candidate for a potential intraocular lens (IOL) implantation procedure;
determining an education level of the candidate patient with respect to IOL implantation based on an artificial intelligence (AI) analysis of the candidate profile data; and
curating education materials for the candidate patient based on the determined education level and based on the candidate profile data.

Example 44. The method of Example 43, wherein the curating the education materials comprises filtering a library of available IOL education materials to exclude materials that are determined to not be relevant to the candidate patient based on the candidate profile data.

Example 45. The method of Example 43 or Example 44, wherein the curating the education materials comprises selecting, from the library of available IOL education materials, materials that address specific ocular conditions identified in the candidate profile data.

Example 46. The method of any of Examples 43-45, wherein the curating the education materials comprises excluding education materials related to presbyopia-correcting IOLs when the candidate profile data indicates the presence of glaucoma.

Example 47. The method of any of Examples 43-46, wherein the curating the education materials comprises including education materials related to astigmatism correction when the candidate profile data indicates that the candidate patient wears multifocal contact lenses.

Example 48. The method of any of Examples 43-47, wherein the curating the education materials is further based on lifestyle data included in the candidate profile data indicating visual usage patterns of the candidate patient, the lifestyle data including at least one of a reading distance, a screen time duration, a driving habit, or an activity level of the candidate patient.

Example 49. The method of Example 48, wherein the lifestyle data includes a device holding distance of the candidate patient with respect to a smart device, the device holding distance being indicative of a near vision demand of the candidate patient.

Example 50. The method of any of Examples 43-49, wherein the curating the education materials comprises including education materials related to a presbyopia-correcting IOL when the candidate profile data indicates that the candidate patient holds a smart device at within a threshold distance from the candidate patient's eye.

Example 51. The method of any of Examples 43-50, wherein the curating the education materials is further based on electronic health record data included in the candidate profile data, the electronic health record data including at least one of a medication list, a prior surgical history, or a medical condition history of the candidate patient.

Example 52. The method of Example 51, wherein the electronic health record data includes pharmacy records indicating that the candidate patient has been prescribed glaucoma medications, and wherein the curating the education materials comprises excluding education materials related to advanced technology IOLs in response to the pharmacy records indicating the glaucoma medications.

Example 53. The method of any of Examples 43-52, wherein the curating the education materials is further based on prior ocular surgical history of the candidate patient included in the candidate profile data.

Example 54. The method of any of Examples 43-53, wherein the determining the education level is based on analyzing questions posed by the candidate patient to an electronic virtual assistant.

Example 55. The method of any of Examples 43-54, further comprising delivering the curated education materials to the candidate patient via at least one of a mobile application, a web-based portal, or a communication link delivered via at least one of a text message or an email.

Example 56. The method of Example 55, wherein the communication link includes a semi-anonymous personalized link requiring the candidate patient to enter a verification credential prior to accessing the curated education materials.

Example 57. The method of any of Examples 43-56, further comprising updating the curated education materials provided to the candidate patient based on additional candidate profile data obtained after an initial delivery of the curated education materials.

Example 58. The method of Example 57, wherein the additional candidate profile data includes at least one of clinical measurements obtained during a pre-operative examination or diagnostic data obtained from at least one imaging device.

Example 59. The method of Example 57 or Example 58, wherein the updating the curated education materials is triggered when a subsequent AI analysis of the additional candidate profile data generates an IOL recommendation that differs from an initial IOL recommendation generated based on the candidate profile data obtained prior to the pre-operative examination.

Example 60. The method of any of Examples 43-59, wherein the curating the education materials is further based on outcome data from previous patient profile data corresponding to prior IOL patients.

Example 61. The method of any of Examples 43-61, wherein the curating the education materials comprises including education materials related to a simultaneous glaucoma surgical procedure when the candidate profile data indicates that the candidate patient has glaucoma, the education materials informing the candidate patient that a glaucoma procedure may be performed concurrently with a cataract surgery procedure.

What is claimed is:

1. A system for selecting an intraocular lens for implantation into an eye of a patient, the system comprising:
a controller having a processor and tangible, non-transitory memory on which instructions are recorded;
wherein the controller is configured to receive data from a smart device operable by the patient, the data received from the smart device indicating at least one input factor related to the patient;
wherein the controller is configured to select, based on the at least one input factor, a chosen machine learning model from a plurality of trained models adaptively trained with a respective training dataset drawn from at least one predefined category, the controller being configured to selectively execute the chosen machine learning model from the plurality of trained models; and
wherein the controller is configured to generate an automated recommendation for the intraocular lens from a group of intraocular lenses based in part on a plurality of input factors related to the patient, including at least one input factor as indicated by the data received from the smart device, and an output generated by the chosen machine learning model.

2. The system of claim 1, wherein the plurality of input factors include the data received from the smart device, the data including usage details of the patient on the smart device, including a viewing distance between the patient and the smart device.

3. The system of claim 2, wherein:
the smart device from which the data is received includes at least one of a mobile phone, smart watch, and tablet; and
the data received from the smart device includes visual performance measurements of the patient made with the smart device.

4. The system of claim 1, wherein the at least one predefined category includes a specific health care provider such that the respective training dataset is drawn exclusively from patient data of the specific health care provider.

5. The system of claim 1, wherein the at least one predefined category includes a specific facility such that the respective training dataset is drawn exclusively from patient data associated with the specific facility.

6. The system of claim 1, wherein the at least one predefined category includes a specific region such that the respective training dataset is drawn exclusively from patient data from the specific region.

7. The system of claim 1, wherein the at least one predefined category includes a specific demographic, including at least one of age, gender, ethnicity, patient motivation and socioeconomic status.

8. The system of claim 1, wherein:
the plurality of input factors include questions of the patient presented to an electronic virtual assistant; and
the electronic virtual assistant is adapted to answer the questions posed by the patient.

9. The system of claim 1, wherein the controller is configured to generate a respective weighted recommendation for each member of the group of intraocular lenses and the chosen machine learning model on generative artificial intelligence.

10. The system of claim 9, wherein the group of intraocular lenses includes:
a first intraocular lens; and
a second intraocular lens adapted to provide improved distance vision and improved intermediate vision relative to the first intraocular lens.

11. The system of claim 1, wherein the plurality of input factors includes preoperative physical data from at least one imaging device, the controller being adapted to receive the preoperative physical data from the at least one imaging device, the at least one imaging device including at least one of a biometer, an OCT device, or a camera system.

12. The system of claim 1, wherein the controller is adapted to select the intraocular lens based in part on at least one comorbidity rule-out marker generated by the controller, including a cornea irregularity marker for the patient.

13. The system of claim 1, wherein the controller is adapted to select the intraocular lens based in part on at least one comorbidity rule-out marker generated by the controller, including an eye dryness marker for the patient.

14. The system of claim 1, wherein the controller is adapted to select the intraocular lens based in part on at least one comorbidity rule-out marker generated by the controller, including a retinal surface irregularity marker for the patient.

15. An apparatus for selecting an intraocular lens for implantation in an eye of a patient, comprising:
a controller having a processor and tangible, non-transitory memory on which instructions are recorded;
wherein the controller is configured to receive data from a smart device operable by the patient, the data received from the smart device indicating at least one input factor related to the patient;
wherein the controller is configured to select, based on the at least one input factor, a chosen machine learning model from a plurality of trained models, the controller being configured to selectively execute the chosen machine learning from the plurality of trained models;
wherein the plurality of trained models are adaptively trained with a respective training dataset drawn from at least one predefined category, the at least one predefined category including a specific health care provider such that the respective training dataset is drawn exclusively from respective patients of the specific health care provider;
wherein the controller is configured to generate an automated recommendation for the intraocular lens from a group of intraocular lenses based in part on a plurality of input factors related to the patient, including at least one input factor as indicated by the data received from the smart device, and an output generated by the chosen machine learning model; and
wherein the plurality of input factors include usage details of the patient on the smart device, including a viewing distance of the smart device from the eye of the patient.

16. The apparatus of claim 15, wherein the at least one predefined category further includes a specific demographic, including at least one of age, gender, ethnicity, patient motivation and socioeconomic status.

17. The apparatus of claim 16, wherein the plurality of input factors include the data received from the smart device, the data including queries generated by the patient and presented to an electronic virtual assistant in the smart device.

18. The apparatus of claim 16, wherein the controller is adapted to select the intraocular lens based in part on at least one comorbidity rule-out marker generated by the controller, the at least one comorbidity rule-out marker including at least one of a cornea irregularity marker, an eye dryness marker, or a retinal surface irregularity marker for the patient.

19. The apparatus of claim 15, wherein:
the controller is configured to generate a respective weighted recommendation for each member of the group of intraocular lenses; and
the group of intraocular lenses includes:
a first intraocular lens; and
a second intraocular lens adapted to provide improved distance vision and improved intermediate vision relative to the first intraocular lens.

20. The apparatus of claim 15, wherein the plurality of input factors include preoperative physical data from at least one imaging device, the controller being adapted to receive the preoperative physical data from the at least one imaging device, the at least one imaging device including at least one of a biometer, an OCT device, or a camera system.
